(19) United States
(12) Patent Application Publication
(10) Pub. No.: US 2019/0126010 A1
(43) Pub. Date: May 2, 2019

(54) SYSTEMS, METHODS, AND DEVICES FOR DELIVERING SUBSTANCES INTO A FALLOPIAN TUBE

(71) Applicant: nVISION MEDICAL CORPORATION, San Bruno, CA (US)

(72) Inventors: Surbhi Sarna, San Francisco, CA (US); Jesus Magana, Redwood City, CA (US); Christina Christman-Skieller, San Bruno, CA (US); David Snow, San Carlos, CA (US)

(21) Appl. No.: 16/172,190

(22) Filed: Oct. 26, 2018

Related U.S. Application Data
(60) Provisional application No. 62/578,168, filed on Oct. 27, 2017.
(60) Provisional application No. 62/599,555, filed on Dec. 15, 2017.

(51) Int. Cl. A61M 25/10; A61B 17/425; A61B 10/02

(52) U.S. Cl. CPC A61M 25/1002; A61B 17/425; A61B 10/0291; A61M 2025/1081; A61M 2025/105; A61M 2025/1079; A61M 2210/1425

(57) ABSTRACT

A system for delivering one or more substances into a Fallopian tube of a patient may include a balloon catheter including a tube having a distal end, a balloon having a first end coupled to the distal end of the tube, and a push wire having a distal end coupled to a second end of the balloon, which may be hollow. The balloon may be movable between an inverted position and an everted position. The balloon catheter may be configured to receive the one or more substances such that the one or more substances may be retained by the balloon, or delivered through the push wire, or both. During eversion, or in the everted position, or both, the one or more substances may be delivered into the Fallopian tube.

### CROSS-REFERENCE TO RELATED APPLICATIONS

This application is a nonprovisional application of, and claims the benefit of priority to, U.S. Provisional Application Ser. No. 62/578,168, filed Oct. 27, 2017, entitled “Devices for Delivering Substances into a Fallopian Tube,” and U.S. Provisional Application Ser. No. 62/599,555, filed Dec. 15, 2017, entitled “Devices for Delivering Substances into a Fallopian Tube,” the entire disclosures of which applications are expressly incorporated by reference herein.

### FIELD

The present disclosure generally relates to Fallopian tube diagnostics and delivery devices that accommodate the anatomical difficulties associated with navigation in the Fallopian tube, and in particular to delivery devices, systems, and methods for delivering substances within the Fallopian tube.

### BACKGROUND

The Fallopian tube is an extremely fragile anatomical lumen. Although medical procedures including research, preventative care, and treatment may require access into the Fallopian tube, the Fallopian tube may be prone to perforation during passage of most devices due to its fragile state. Access to the Fallopian tube may be necessary in medical procedures such as diagnostic procedures related to cancer diagnosis and treatment, in vitro fertilization (IVF), and/or artificial insemination (AI), or other therapeutic delivery devices. Some types of AI may include intrauterine tuboperitoneal insemination (IUTPI), which is an AI technique that involves injection of washed sperm into both the uterus and Fallopian tubes. Intratubal insemination (ITI) is an AI technique that involves injection of washed sperm into the fallopian tube. Gamete intrafallopian transfer is an AI technique where both eggs and sperm are mixed outside the woman's body and then immediately inserted into the Fallopian tube where fertilization takes place.

The introduction of diagnostic, treatment, and fertility devices to introduce substances into the Fallopian tube during medical procedures may be challenging due to the fragile structure of the Fallopian tube described above. Thus, there is a need for devices and processes to allow introduction of substances into the Fallopian tube during medical procedures in a less invasive and controlled fashion, and without the need for open or laparoscopic surgery.

It is with respect to these and other considerations that the present improvements may be useful.

### SUMMARY

This Summary is provided to introduce a selection of concepts in a simplified form that are further described below in the Detailed Description. This Summary is not intended to necessarily identify key features or essential features of the claimed subject matter, nor is it intended as an aid in determining the scope of the claimed subject matter.

According to an exemplary embodiment of the present disclosure, a system for delivering one or more substances into a Fallopian tube of a patient may include a balloon catheter including a tube having a distal end. A balloon may have a first end coupled to the distal end of the tube. A push wire may have a distal end coupled to a second end of the balloon. The balloon may be movable between an inverted position and an everted position by actuation of the push wire. The balloon catheter may be configured to receive the one or more substances such that the one or more substances are retained by the balloon, the push wire, or both, and may advance the push wire to evert the balloon such that the balloon extends distally of the distal end of the tube. In the everted position the one or more substances may be delivered into the Fallopian tube.

In various of the foregoing and other embodiments of the present disclosure, the balloon catheter may receive the one or more substances at the distal end of the tube and the first end of the balloon when the balloon is in the inverted position. The push wire may be hollow, and the one or more substances may be received into the proximal end of the push wire. The balloon catheter may receive a first substance at the distal end of the tube and the first end of the balloon, and the push wire may be hollow such that a second substance may be received into the proximal end of the push wire. The one or more substances may at least partially coat an inner surface of the balloon when the balloon is in the inverted position. The balloon catheter may include a filament attached to the distal end of the push wire, or the second end of the balloon, or both. The filament may be configured to absorb at least a portion of the one or more substances. The filament may be configured to receive a first substance, and the balloon may be configured to receive a second substance different from the first substance. The one or more substances may be at a temperature different than a temperature of the patient. The one or more substances may be any of a radiopaque marker, a radiopaque marking material, a gel, a chemotherapeutic, a fertility therapeutic, an antibiotic, an anti-inflammatory agent, a tissue protecting substance, a dissolvable object, an impermeable object, or a radiation delivering object, or combinations thereof.

According to an exemplary embodiment of the present disclosure, a system for depositing one or more substances in a Fallopian tube of a patient may include a balloon catheter to receive the one or more substances. The balloon catheter may include a tube having a distal end and a balloon having a first end coupled to the distal end of the tube. A push wire for advancement may evert the balloon. The push wire may have a distal end coupled to a second end of the balloon, and the balloon may be movable between an inverted position and an everted position by actuation of the push wire. The one or more substances may be retained by the balloon, the push wire, or both. The one or more substances may be depositable into the Fallopian tube in the everted position of the balloon.

In various of the foregoing and other embodiments of the present disclosure, prior to receiving the one or more substances into the balloon catheter, the balloon may be inflatable in the inverted position by an inflation fluid. The balloon may be evertible such that at least a portion of the balloon may be extended distally of the distal end of the tube. After receiving the one or more substances into the balloon catheter, the push wire may be retractable such that the balloon is re-inverted and positionable proximal of the distal end of the tube such that the one or more substances are retained by the balloon. The balloon may be positionable within a sheath such that the balloon may be extendable from a distal end of the sheath during eversion and may support the balloon during re-inversion. The balloon catheter may include a filament attached to the distal end of the push wire, or the second end of the balloon, or both. The filament may be configured to absorb at least a portion of the one or more substances. The filament may be configured to receive a first of the one or more substances substance, and the balloon may be configured to receive a second of the one or more substances different from the first substance. The push wire may be hollow, and the one or more substances may be received into the proximal end of the push wire. The one of more substances may be any of a radiopaque marker, a radiopaque marking material, a gel, a chemotherapeutic, a fertility therapeutic, an antibiotic, an anti-inflammatory agent, a tissue protecting substance, a dissolvable object, an impermeable object, a radiation delivering object, or a combination thereof.

According to an exemplary embodiment of the present disclosure, a method for depositing one or more substances in a Fallopian tube of a patient may include receiving the one or more substances into a balloon catheter. The balloon catheter may include a tube having a distal end. A balloon may have a first end coupled to the distal end of the tube. A push wire may have a distal end coupled to a second end of the balloon. The balloon may be movable between an inverted position and an everted position by actuation of the push wire. The one or more substances may be retained by the balloon, the push wire, or both. The push wire may be advanced to evert the balloon to the everted position such that the balloon may extend distally of the distal end of the tube. The one or more substances may be deposited into the Fallopian tube in the everted position of the balloon.

In various of the foregoing and other embodiments of the present disclosure, the balloon catheter may receive the one or more substances at the distal end of the tube and the second end of the balloon when the balloon is in the inverted position. The balloon catheter may receive a first of the one or more substances at the distal end of the tube and the second end of the balloon. The push wire may be hollow such that a second of the one or more substances may be received into the proximal end of the push wire. The one or more substances may at least partially coat an inner surface of the balloon when the balloon is in the inverted position. The push wire may be advanced to position the balloon in the everted position prior to receiving the one or more substances in the balloon catheter, such that a surface of the balloon contacting an inner surface of the Fallopian tube in the everted position may be free of a coating of the one or more substances.

### BRIEF DESCRIPTION OF THE DRAWINGS

Non-limiting embodiments of the present disclosure are described by way of example with reference to the accompanying figures, which are schematic and not intended to be drawn to scale. In the figures, each identical or nearly identical component illustrated is typically represented by a single numeral. For purposes of clarity, not every component is labeled in every figure, nor is every component of each embodiment shown where illustration is not necessary to allow those of ordinary skill in the art to understand the disclosure. In the figures:

FIG. 1 illustrates a cross-sectional view of a Fallopian tube with the uterotubal junction (UTJ) that connects the uterus to the ovaries;

FIG. 2 illustrates a schematic of a hysteroscope for deploying an exemplary embodiment of a catheter in accordance with the present disclosure;

FIG. 3 illustrates an exemplary embodiment of a proximal introducer catheter in accordance with the present disclosure;

FIG. 4A illustrates a cross-sectional view of an exemplary embodiment of a device for delivering a substance to a Fallopian tube in accordance with the present disclosure;

FIG. 4B illustrates a detailed view of FIG. 4A in accordance with the present disclosure;

FIG. 4C illustrates a cross-sectional view of an exemplary embodiment of a catheter having a through lumen for delivering a substance to a Fallopian tube in accordance with the present disclosure;

FIGS. 5A-5E illustrate an exemplary embodiment of a method for capturing and delivering a substance to a Fallopian tube in accordance with the present disclosure;

FIG. 6 illustrates a cross-sectional view of an exemplary embodiment of a catheter in accordance with the present disclosure, with kinking condition of an everted balloon that may occur due to a lack of adequate support of the sheath;

FIG. 7A illustrates a cross-sectional view of an exemplary embodiment of a ball tip everting balloon catheter prior to deployment in accordance with the present disclosure;

FIG. 7B illustrates a cross-sectional side view of an exemplary embodiment of a ball tip everting balloon catheter;

FIG. 8A illustrates a cross-sectional side view of an exemplary embodiment of a balloon catheter in accordance with the present disclosure;

FIG. 8B illustrates the balloon catheter of FIG. 8A in accordance with the present disclosure;

FIG. 8C illustrates the balloon catheter of FIG. 8A and an exemplary embodiment of a high-pressure tubing reservoir and inflation device in accordance with the present disclosure;

FIG. 9 illustrates a cross-sectional side view of an exemplary embodiment of a balloon catheter configured in accordance with the present disclosure;

FIG. 10 illustrates a side view of an exemplary embodiment of a balloon catheter in accordance with the present disclosure;

FIG. 11A illustrates a cross-sectional view of an exemplary embodiment of a handle of the catheter of FIG. 10 in accordance with the present disclosure;

FIG. 11B is a detail view illustrating an exemplary embodiment of a gear system in the handle portion of the catheter of FIG. 11A in accordance with the present disclosure;

FIG. 11C illustrates a perspective view of an exemplary embodiment of a linear rack ratcheting assembly in accordance with the present disclosure;

FIG. 11D illustrates a side view of an exemplary embodiment of a drop key-click of the linear rack ratcheting assembly of FIG. 11C in accordance with the present disclosure;

FIG. 11E illustrates a side view of an exemplary embodiment of a gear jam in accordance with the present disclosure;

FIG. 12 illustrates a cross-sectional side view of an exemplary embodiment of a balloon catheter in accordance with the present disclosure;

FIG. 13 illustrates a cross-sectional side view of an exemplary embodiment of a balloon catheter in accordance with the present disclosure;

FIG. 14 illustrates a cross-sectional side view of an exemplary embodiment of a balloon catheter with a sheath and a balloon inverted in accordance with the present disclosure;

FIG. 15A illustrates a side perspective view of an exemplary embodiment of a string filament with a series of printed indicia in accordance with the present disclosure;

FIG. 15B illustrates a side perspective view of an exemplary embodiment of a string filament with a series of knots as indicia in accordance with the present disclosure;

FIG. 16 illustrates an eversion of an exemplary embodiment of a balloon in accordance with the present disclosure;

FIG. 17A illustrates a cross sectional view of an exemplary embodiment of a balloon in accordance with the present disclosure;

FIG. 17B illustrates a cross sectional view of an exemplary embodiment of a balloon in accordance with the present disclosure;

FIG. 18A illustrates a cross-sectional view of an exemplary embodiment of an everting balloon catheter in a deflated state in accordance with the present disclosure;

FIG. 18B illustrates the everting balloon catheter of FIG. 18A in an inflated state in accordance with the present disclosure.

### DETAILED DESCRIPTION

The present disclosure is not limited to the particular embodiments described herein. The terminology used herein is for the purpose of describing particular embodiments only, and is not intended to be limiting beyond the scope of the appended claims. Unless otherwise defined, all technical terms used herein have the same meaning as commonly understood by one of ordinary skill in the art to which the disclosure belongs.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the disclosure. The upper and lower limits of these smaller ranges may independently be included or excluded in the range, and each range where either, neither or both limits are included in the smaller ranges is also encompassed within the disclosure, subject to any specifically excluded limit in the stated range. Where the stated range includes one or both of the limits, ranges excluding either or both of those included limits are also included in the disclosure.

As used herein, the singular forms “a,” “an,” and “the” are intended to include the plural forms as well, unless the context clearly indicates otherwise. It will be further understood that the terms “comprises” and/or “comprising,” or “includes” and/or “including” when used herein, specify the presence of stated features, regions, steps elements and/or components, but do not preclude the presence or addition of one or more other features, regions, integers, steps, operations, elements, components and/or groups thereof.

As described above, performing procedures which require access into the Fallopian tubes without undergoing a surgical procedure may be challenging. Exemplary embodiments of the present disclosure include systems, methods and devices for introducing substances into the Fallopian in a less invasive procedure. Anatomically, the ovaries are in close proximity to the fimbria at the region of the distal opening or distal os of the Fallopian tube. Eggs released by the ovary may be gathered by the fimbria and transported through the Fallopian tube to the uterus. With ovarian cancer, cells may be deposited in the Fallopian tube, which may eventually migrate into the uterus. Cell samples obtained from the uterus may be analyzed to detect ovarian malignancy; however, the incidence of migration of ovarian cancer cells into the uterus may be too low to render uterine sampling a reliable diagnostic test for ovarian malignancy and/or abnormality. Reliable access to the Fallopian tube without damaging the fragile structure, for diagnostic sampling and delivery of substances is therefore desirable.

As described above, the fragility of the Fallopian tube may result in perforation during a medical procedure. As such, safe introduction of a diagnostic device into the Fallopian tube may be difficult with known devices. Referring now to FIG. 1, a Fallopian tube 1 of a patient may extend from a proximal os 3 to a uterus, connecting at a uterotubal junction (UTJ) 2, to a distal os 5 and connecting to ovaries 6. A perforation may occur at the UTJ 2, which is a constriction occurring distal to the proximal os 3 (e.g., opening) of the Fallopian tube. For example, in some patients the UTJ 2 may be approximately 1 cm distal of the proximal os 3. In some patients, the body lumen size at this constriction may be as small as approximately 0.3 mm or 0.5 mm, while the body lumen size of the Fallopian tube adjacent to the UTJ may be approximately 1 mm. The Fallopian tube is curved, and the soft tissue of the tube is in a naturally collapsed state, which may result in multiple constrictions as passage is attempted. Perforation may occur at the uterotubal junction (UTJ), a constriction that occurs approximately 1 cm distal to the proximal os (opening) of the Fallopian tube in the uterus. The UTJ may present a downward bend in the Fallopian tube. The lumen size of the Fallopian tube adjacent to the uterotubal junction may be approximately 1 mm.

Although systems and devices are described in the present disclosure for accessing a Fallopian tube of a patient, it is understood that the systems and devices may be utilized for other body lumens having challenging navigation, including but not limited to blood vessels, arteries, and other vasculature, ducts, tracts, body lumen, and the like.

Exemplary embodiments of a catheter for introducing substances into a Fallopian tube for minimally invasive procedures may include any of the following: (1) access to the proximal os of the Fallopian tube via an intrauterine approach; (2) advance of an introducer catheter to cannulate the proximal os; (3) use of a second catheter inside the introducer catheter to track inside the Fallopian tube. An inflated balloon at the end of the second catheter is advanced across the proximal portion of the Fallopian tube and is everted further into the Fallopian tube; (4) a substance may be released within the Fallopian tube simultaneously with and/or subsequently to balloon eversion; (5) the surface of the balloon may have a coating that contacts and coats the intraluminal surface of the Fallopian tube, which coating may be a released substance from the catheter; and (6) the balloon may be retracted and removed from the Fallopian tube.

Embodiments of an exemplary catheter may be configured for insertion into the Fallopian tube (see FIG. 1). Embodiments of the catheter are also described in U.S. patent application Ser. No. 14/764,710, filed Jul. 30, 2015, entitled “Methods and Devices for Fallopian Tube Diagnostics,” U.S. patent application Ser. No. 15/053,568, filed Feb. 25, 2016, entitled “Methods and Devices for Fallopian Tube Diagnostics,” and U.S. patent application Ser. Nos. 15/998,507 and 15/998,501, filed Aug. 16, 2018, entitled “Systems, Methods, and Devices for Fallopian Tube Diagnostics,” which are herein incorporated by reference in their entireties. The Fallopian tube has a curvature (e.g., having a tortuous pathway), and the soft tissue of the tube may be collapsible, thereby resulting in multiple constrictions as passage is attempted. As described above, this may be particularly true at the uterotubal junction (UTJ), which may be muscular and therefore more prone to perforation by insertion of medical instruments. In some patients, the UTJ may also present a downward bend with a lumen size at the constriction that may be as small as approximately 0.3 mm or 0.5 mm, while the body lumen size of the Fallopian tube adjacent to the UTJ may be approximately 1 mm.

In at least one embodiment of the present disclosure, an elongated balloon that is initially inverted into a catheter lumen may be deployable. The balloon may partially evert to enter a proximal end of the Fallopian tube, e.g., the UTJ, thereby cannulating the proximal os. The balloon may evert upon pressurization of the balloon from inside the catheter so that an unrolling mechanism of the eversion creates a path through the Fallopian tube regardless of tortuosity or constriction in the Fallopian tube. In some embodiments, the balloon may evert by a push wire advancement, which may be in concert with pressurization. Up to a great majority of the length of the balloon may be substantially inelastic, such that the balloon does not substantially expand and over-dilate the Fallopian tube as it everts. Balloon expansion may burst or otherwise damage or injure the Fallopian tube. However, exemplary embodiments may also incorporate an elastic distal balloon end expandable to seal the distal os upon retraction of the distal balloon. In embodiments, the device may have a balloon having a sufficient rigidity to cannulate the Fallopian tube and sufficient flexibility for navigation through the tortuous path of the Fallopian tube to minimize potential damage or injury. In some embodiments, the device may include support elements for cannulating the Fallopian tube so that the balloon may not collapse at the proximal os.

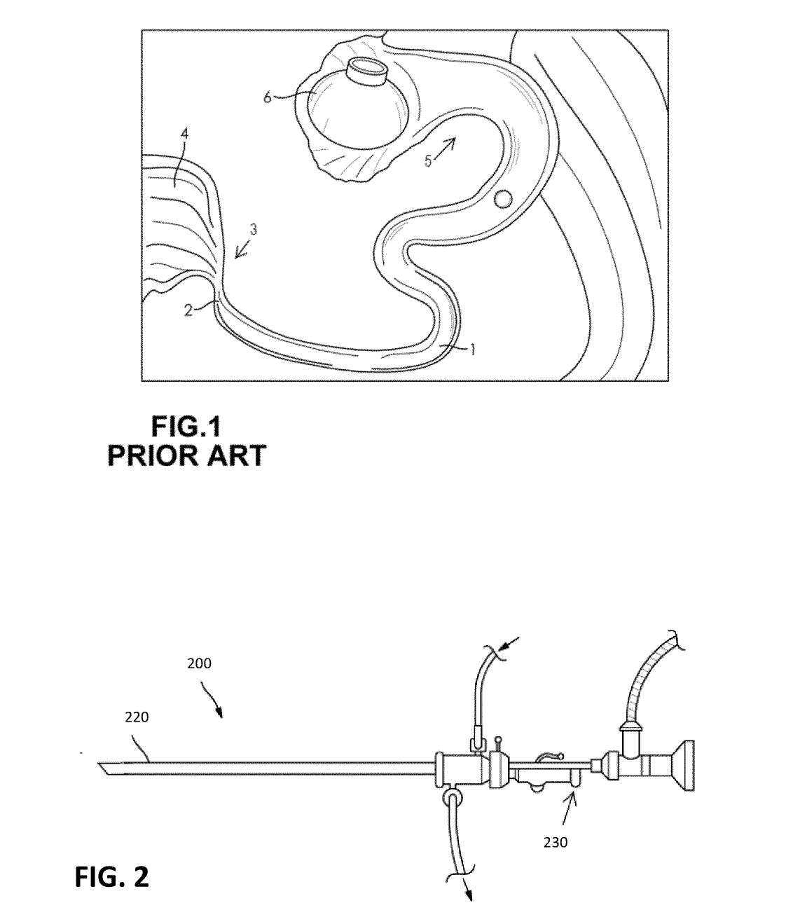

Exemplary embodiments of systems and methods of the present disclosure may include positioning, and deployment of, a distal end of a catheter. In some embodiments, a catheter distal end may be deliverable to a proximal end of the Fallopian tube by a hysteroscope. In some embodiments, the hysteroscope may be an exemplary hysteroscope (e.g., FIG. 2). Regardless of the mode of deployment, a retracted portion of a catheter may be extendable to contact the interior wall of the Fallopian tube. It has been surprisingly found that the act of extending a portion of the catheter may remove a sufficient sampling of cells and/or tissue from the Fallopian tube wall to perform histological and/or cytological evaluation. For example, at least a portion of a length of the balloon may contact the Fallopian tube for sample collection. In some embodiments, a majority of the length of the balloon may be substantially inelastic such that the balloon does not substantially expand and over-dilate the body lumen (e.g., Fallopian tube) as it everts. In some embodiments, the balloon may be sized such that the body lumen does not expand or over-dilate as the balloon everts. As described above, balloon expansion may burst or injure the subject's body lumen. According to some embodiments and as discussed above with regard to the exemplary balloon catheter, the balloon may be extendable by eversion from a catheter only longitudinally into the body lumen such that the balloon does not substantially expand and dilate the lumen as the balloon everts or is extended into the body lumen (e.g., the Fallopian tube). In some embodiments, the balloon may be extendable longitudinally into the body lumen, where a diameter of an inflated balloon may be up to approximately 10-15% greater than a diameter of a Fallopian tube. Radial expansion of the balloon may be limited or controlled by the majority of the length of the balloon being substantially inelastic. It is appreciated that portions of a balloon that are not intended to be inserted within a lumen structure can be elastomeric and therefore may be expandable in diameter and compliant rather than substantially inelastic. Such a hybrid balloon may be well-suited in embodiments when a seal is desired with the UTJ. Exemplary of situations when a seal is desired may include irrigation of the lumen, filling the lumen with an imaging contrast, diagnosing obstructions, and/or topical contact with a therapeutic agent, such as a chemotherapeutic or an antibiotic.

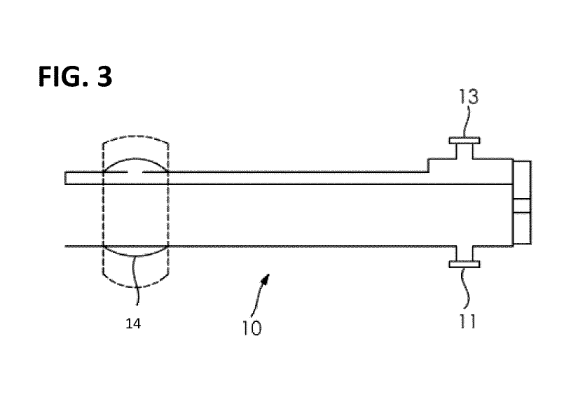

An exemplary embodiment of a catheter, as described below, may be introduced into the uterus of a patient using an operating hysteroscope 200, an example of which is shown in FIG. 2. An operating hysteroscope 200 may include one or more ports. One or more ports may provide irrigation to distend the uterus and allow endoscopic visualization, and port 230 may provide access to a working channel 220 which may allow instruments and/or catheters to be advanced distally of the hysteroscope. A proximal introducer catheter 10 (see, e.g., FIG. 3) may be advanceable through a working channel of the operating hysteroscope 200, and may be used to cannulate the proximal os of a Fallopian tube. A balloon 14 on the proximal introducer catheter 10 may be inflated to occlude the proximal os, and the everting sleeve catheter may be advanceable through the proximal introducer catheter 10 into the proximal portion of the Fallopian tube. The sleeve/balloon element 14 may be fully everted, and the inflated balloon tip may be pulled back to seal the distal os. Irrigation may be introduced via a port 11, and aspirated via the irrigation port 11 on the proximal introducer catheter 10, to collect the sample. Irrigation may also be introduced through both the everting sleeve catheter and the proximal introducer catheter, followed by aspiration through one or both ports (11, 13) of the proximal introducer catheter.

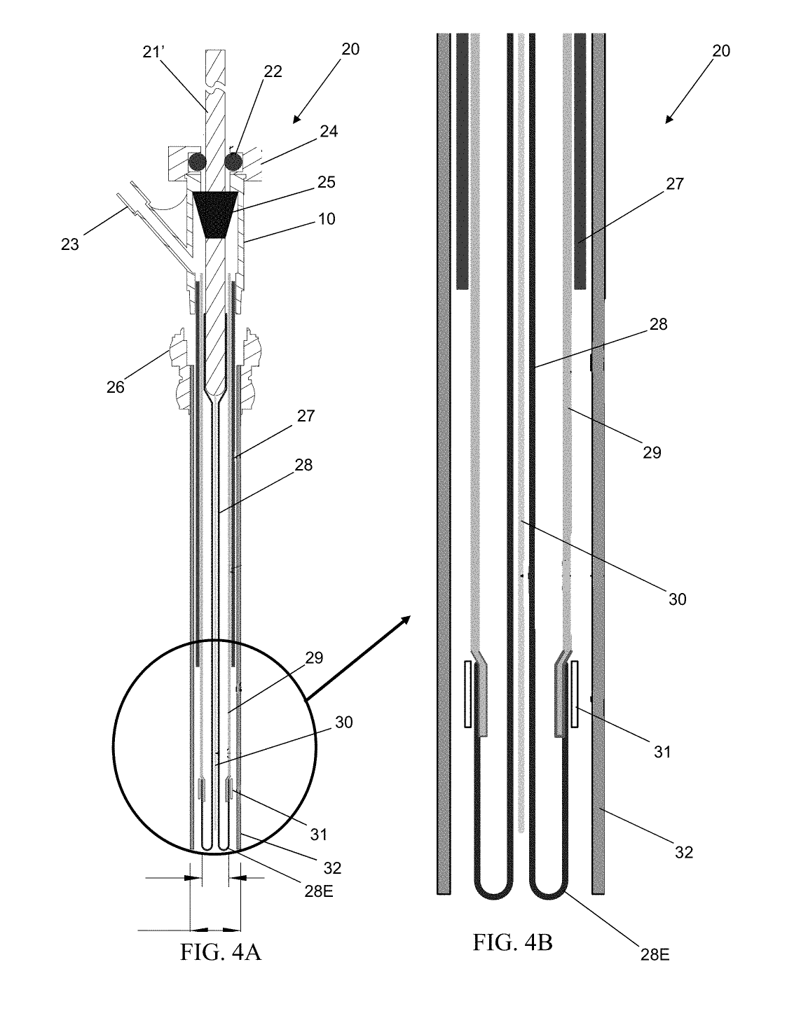

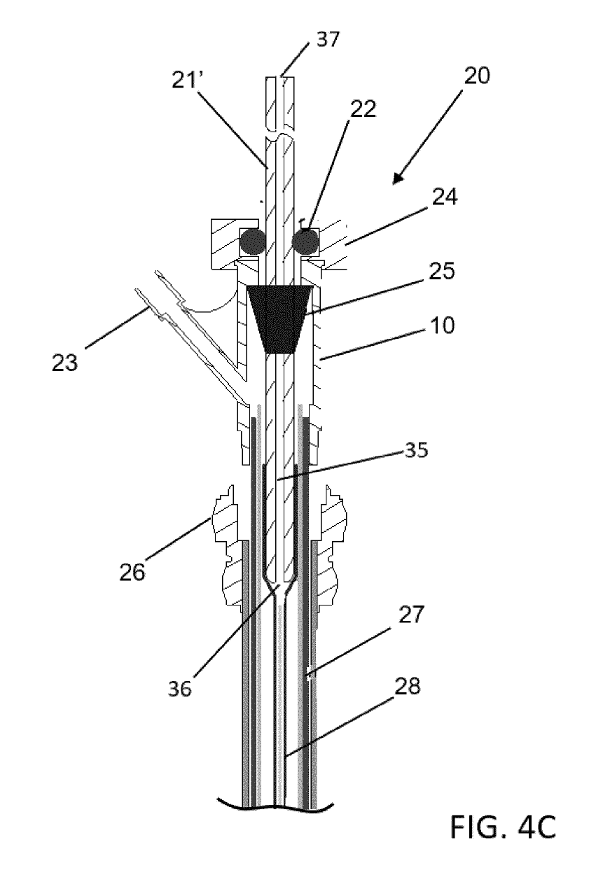

Referring now to FIGS. 4A-4B, FIG. 4A illustrates an exemplary embodiment of a catheter 20 for delivering a substance to a Fallopian tube, and FIG. 4B is an expanded detailed view of the distal end of FIG. 4A. As shown in FIG. 4A a sheath 32 may substantially form a cylinder with an open distal end and a proximal end connected to a sheath knob 26. The sheath 32 may be a catheter and may be configured to slide longitudinally along a tube 27 attached to an operating port 10 with a Y-connector 23 for injection of an inflation fluid, such as gas, liquid, or air, or combinations thereof, into the balloon 28. In some embodiments, the tube 27 may be formed of a rigid material, such as metal including stainless steel. A push wire 21 may be slideably connected to the operating port 10 and may extend through an O-ring seal 22 at the center axis of a seal knob 24 joined to the operating port 10. In embodiments, the O-ring seal 22 may be formed of a silicone material. A conical valve 25 may act as a seal in the operating port 10, e.g., by concentrically surrounding and sealing around the push wire 21. A tube 29 may extend from the operating port 10, and may lie within and be concentric to the tube 27. In embodiments, the tube 29 may be formed of a material such as nylon. In the inverted position, the distal end of the tube 29 may be connected to the leading distal edge of the balloon 28, and the proximal end of the balloon 28 may surround and be sealed to the push wire 21, thereby forming a sealed cavity having a substantially toroidal or doughnut shape.

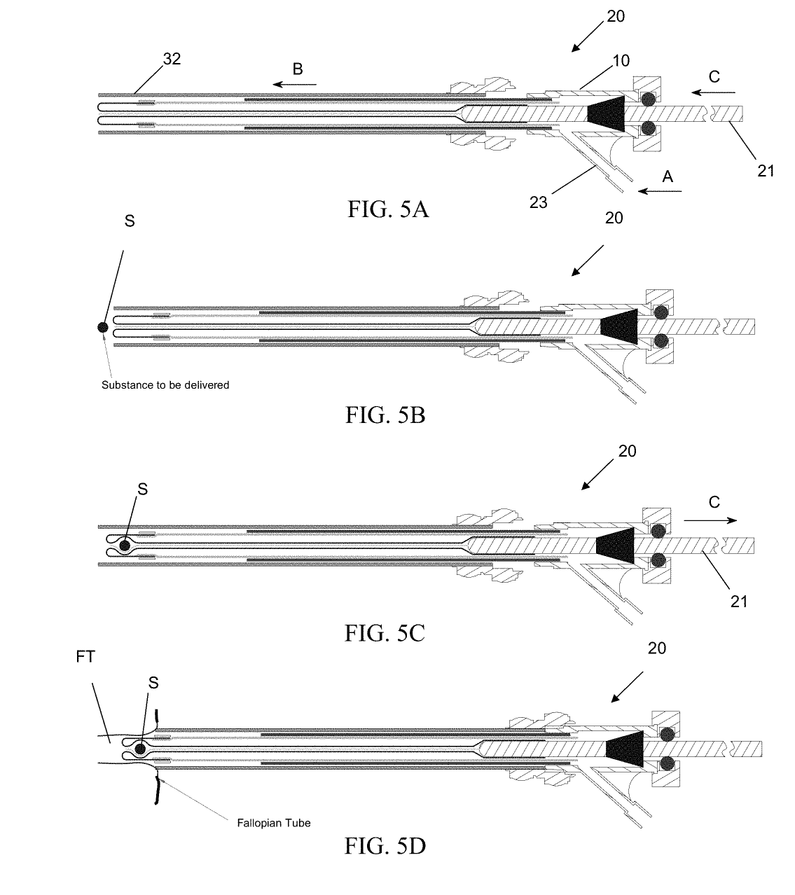

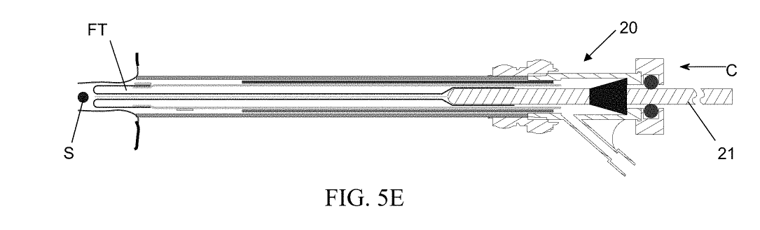

In some embodiments, a suture, or filament 30 may be extendable from the tip of the push wire 21, and may extend through the center of the toroid (doughnut) formed by the inverted balloon 28, although embodiments without a suture or filament are also envisioned. The filament 30 may provide enhanced visibility of the motion of the balloon 28 during deployment. In some embodiments, markers disposed along the length of the filament 30 may aid in visibility to the user. The filament 30 may also improve the ease of insertion of a substance “S” into the distal end. For example, in embodiments where the substance S is delivered via a syringe having a blunt needle tip as described below, the filament 30 may be used to align the needle tip and guide the tip into the balloon 28. The substance “S” may be retained in the balloon in an inverted position prior to insertion in the patient (see e.g., FIG. 5C), and may be held by an inner surface of the balloon in the inverted position. In some embodiments, surface features on the balloon surface, such as wrinkles, overlaps, or folds, may also aid in retaining the substance “S”. In some embodiments, the balloon may be partially or wholly pressurized to hold the substance “S” in the desired positioned.

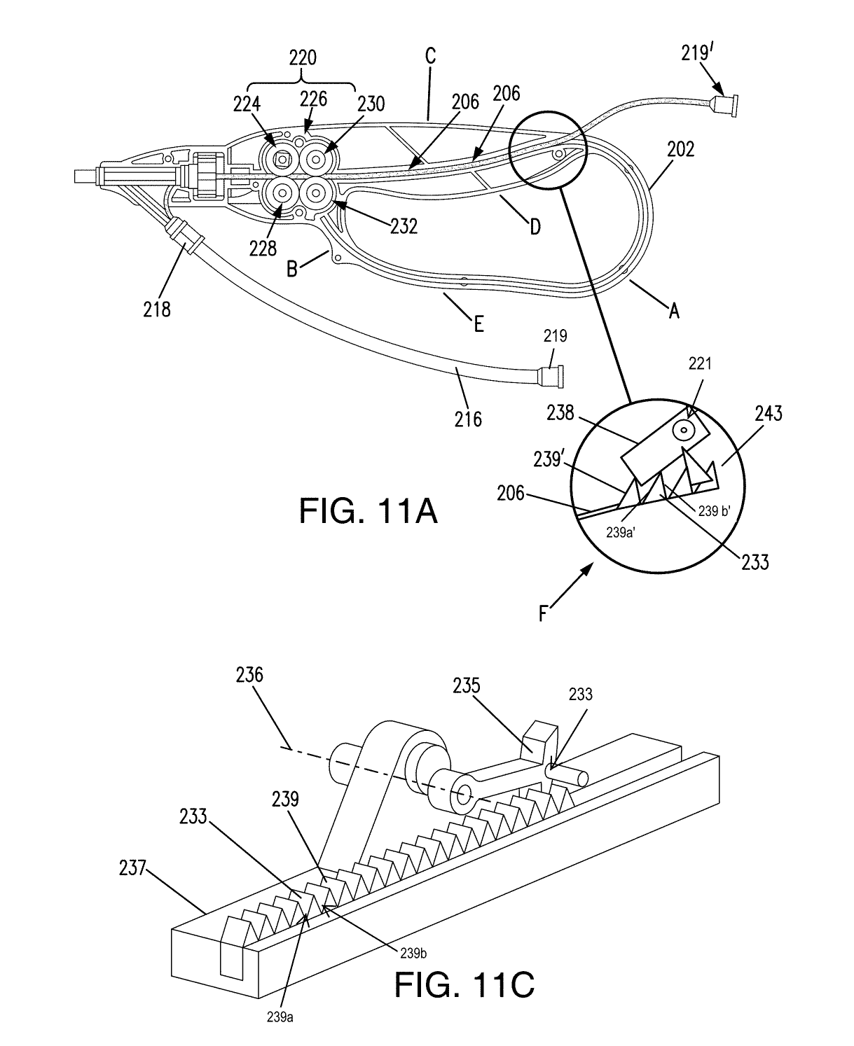

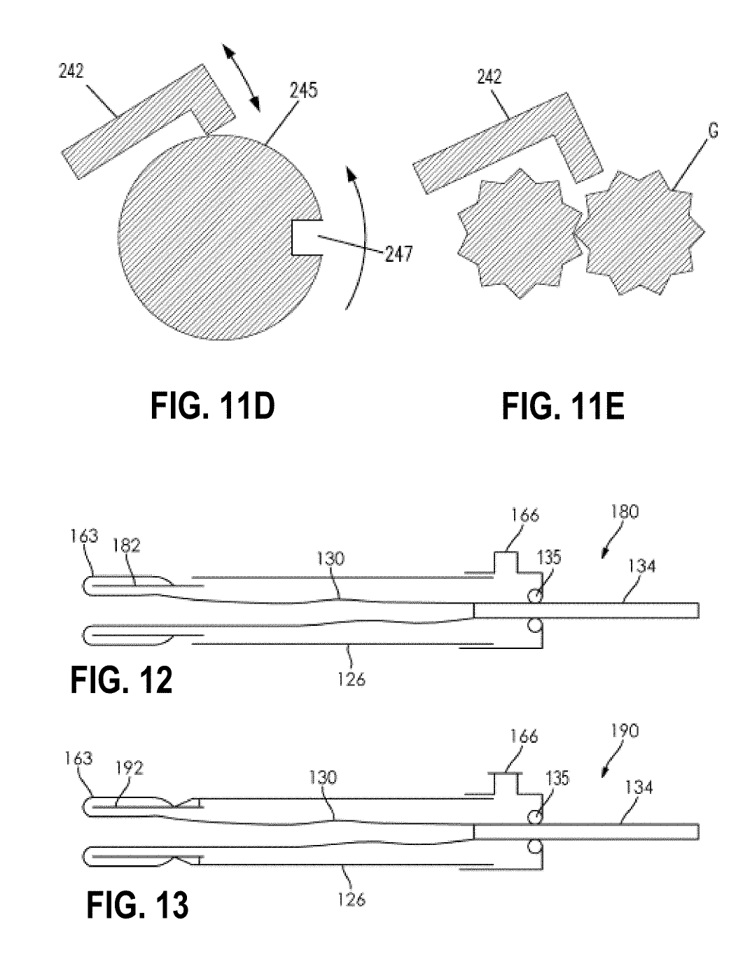

FIG. 4C illustrates a proximal end of the catheter 20 of FIG. 4A for delivering a substance to a Fallopian tube. The push wire 21′ may be slideably connected to the operating port 10 and may extend through O-ring seal 22 at the center axis of a seal knob 24 joined to the operating port 10. In embodiments, the push wire 21′ may include a lumen 35 extending through the push wire 21′, so that the push wire 21′ may have a hollow tubular structure. The push wire 21′ may define a distal opening 36 and a proximal opening 37 of the lumen 35. In some embodiments, the outer surface of the push wire 21′ may be substantially sinusoidal, and/or have a textured (e.g., non-smooth) surface. A textured surface of the push wire 21′ may increase a strength of the long hollow wire, to minimize and/or eliminate potential collapse in response to advancement, and may provide a rack-like surface that can engage with a pawl for fine and precise control and move, which is described in further detail below with respect to FIGS. 11A-11E. The distal end of the balloon 28 in the everted position may be sealed around the push wire 21′ at the distal opening 36 such that the distal opening 36 of the lumen 35 remains unobstructed. The balloon 28 may remain at least partially in fluid communication with the operating port 10 forming a sealed cavity with a toroidal or doughnut shape, e.g., as shown in FIG. 4A.

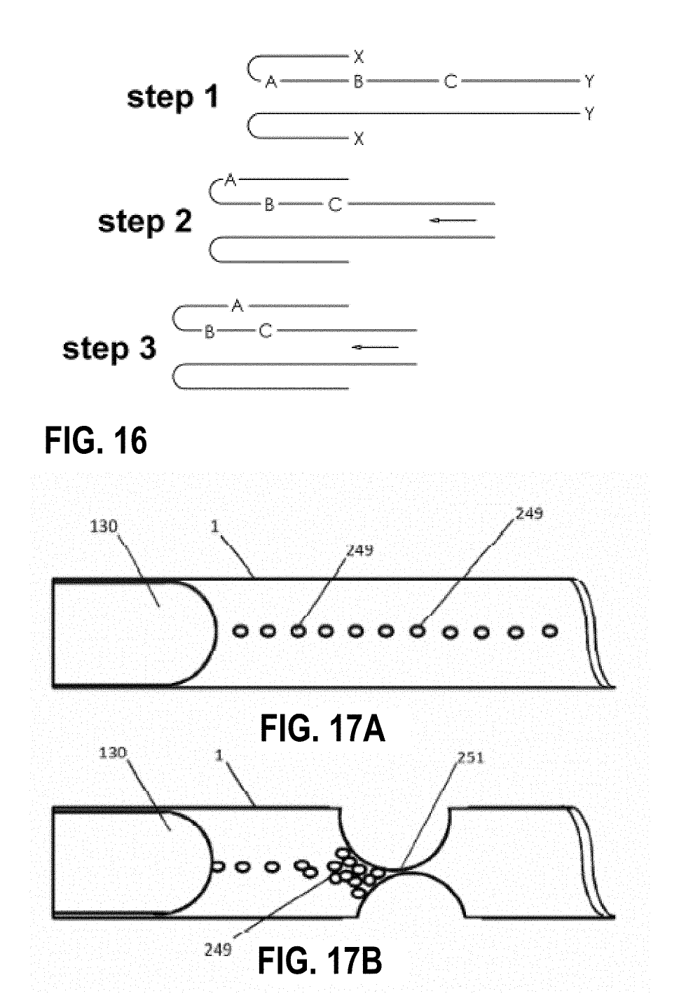

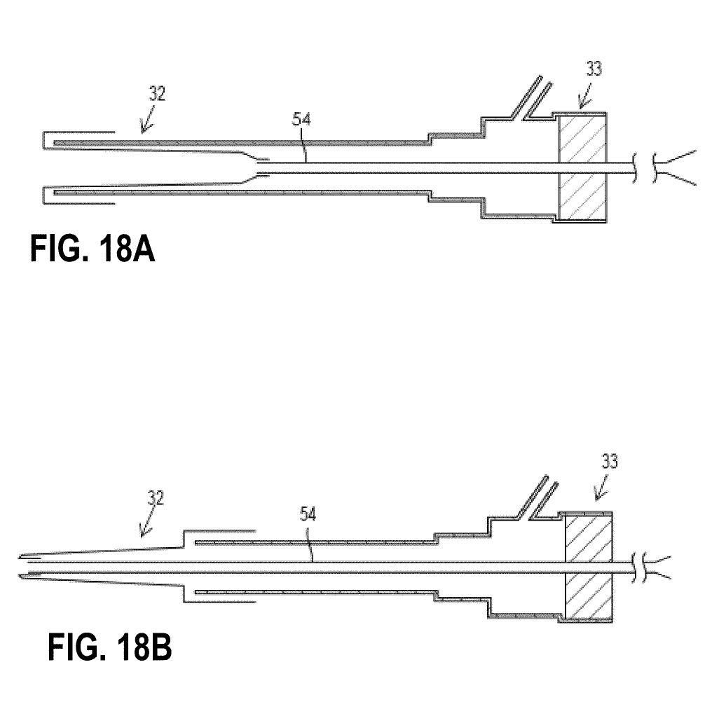

The distal opening 36, the lumen 35, and/or the proximal opening 37 may be at least partially in fluid communication with an internal channel formed by a surface of the balloon 28 that is an inner surface when the balloon 28 is in an inverted position in the catheter. In embodiments, substances for delivery into the Fallopian tube, including but not limited to markers, gels, chemotherapeutics, fertility therapeutics, or antibiotics, may be injected into the lumen 35 via the proximal opening 37, passed through the lumen 35, and dispersed from the distal opening 36. When the balloon 28 is in an inverted position, for example as shown in FIG. 18A, a substance may be dispersed, or ejected, from the lumen 35 into an internal channel formed by the surface of the balloon 28 that is the inner surface when the balloon 28 is in an inverted position. The substance may coat the balloon inner surface, upon which during eversion becomes an outer surface of the balloon (see e.g., FIG. 16), to contact an inner surface of the Fallopian tube when the balloon is everted. In some embodiments, a substance may be injected into the lumen 35 via the proximal opening 37, passed through the lumen 35, and ejected from the distal opening 36 when the balloon 28 is in an everted position. When the balloon 28 is in a fully everted position, for example, as shown in FIG. 18B, the distal opening 36 of the through lumen 35 may be extended distally of the distal end of the catheter 20. Substances dispersed from the distal opening 36 of the lumen 35 of the push wire 21′ when the balloon 28 is in an everted position may be directly applied within the Fallopian tube and directly to the inner surface of the Fallopian tube. This may allow for more precise delivery of substances to a desired location within the Fallopian tube for improved patient treatment.

It is understood that the lumen 35 and associated components for delivering a substance to a Fallopian tube, as described above with regards to FIGS. 4C and 18B, may also be applied to other exemplary embodiments described herein. FIG. 18A illustrates an exemplary embodiment of a catheter according to the present disclosure. It is understood that a hollow push wire 54 of FIG. 18A may be incorporated into exemplary embodiments described herein without limitation to the reference numerals used for the various embodiments.

In some embodiments, a split sample of different substances may be deliverable into the Fallopian tube by applying a first substance to the filament 30 and capturing a second substance with the balloon 28. The filament 30 may absorb, and/or be coated by the first substance, to retain the first substance until delivery in the patient. To minimize cross-contamination of the substances prior to delivery to the patient, the filament may have a length less than a balloon, so that in an inverted position, a distal tip of the filament is proximal to a distal tip of the balloon and/or catheter shaft. To apply a first substance to the balloon surface, the balloon may be at least partially everted up to the distal tip of the suture, so that the inner surface of the balloon is positioned as the outer surface of the balloon, and coated and/or dipped in the first substance. To impregnate the filament with a second substance, the filament may be extended to an exposed position from the balloon to be coated and/or dipped in the second substance. After coating of the first substance on the balloon, and the second substance on the filament, the balloon may be re-inverted, e.g., so that the first substance is disposed on the inner surface of the balloon, and the filament coated in the second substance is disposed within the balloon.

It is understood that when the balloon is everted and re-inverted, the sheath (e.g., sheath 32) may provide support to the balloon to minimize and/or avoid kinking or collapse of the balloon. For example, as shown in FIG. 6, when the balloon lacks sufficient support, a portion of the balloon may kink or collapse, which may make it difficult to achieve, or may altogether prevent, reinversion. The sheath may remain stationary during eversion and/or re-inversion, although in some embodiments, the sheath may be movable along its longitudinal axis to extend and/or retract to support the balloon and the received substance during re-inversion.

In some embodiments, the filament may be detached or detachable from the balloon and/or the push rod, e.g., as a separate component. The filament may be separately impregnated and/or coated with a substance and disposed in the balloon for delivery to the patient. When the balloon is everted, the filament may be disposed in the patient, and may be positioned to deliver a drug therapy over an extended period of time. For example, the suture or filament may be impregnated and/or coated with a substance having a delayed or gradual release component. In some embodiments, the filament may be evacuated naturally by the patient (e.g., due to ciliated motion in the Fallopian tube and/or during menstruation), and in other embodiments, the filament may be removable by a medical profession, e.g., via a hysteroscope and graspers.

In some embodiments, inflation fluid for the balloon 28 may be chilled (e.g., at a predetermined temperature different (lower) than a temperature of the balloon catheter) to a desired temperature to maintain the quality of the substance, e.g., a sperm sample or other treatment substance including temperature-controlled component, prior to injection into the Fallopian tube. For example, a substance may be at a temperature different than a patient to preserve the substance until delivery. In some embodiments, inflation fluid may be warmed (e.g., at a temperature different (above) a predetermined chilled temperature) to help to thaw the temperature-controlled substance, or chilled to maintain the temperature of the temperature-controlled substance, or combinations thereof.

In some embodiments, the balloon 28 and/or the filament 30 may be pre-coated with substances that prepare the substance for treatment, prepare the Fallopian tube for receiving the substance, promote sperm motility, or improve the conditions for fertilization within the Fallopian tube, or combinations thereof. Substances that enhance the health of sperm and promote motility illustratively include substances that decrease reactive oxygen species (ROS), Percoll, and Nicodenz. ROS scavengers illustratively include superoxide dismutase (SOD), L-cysteine, and thioredoxin. Substances that enhance the ability of sperm to bind illustratively include Fe2/Asc to introduce lipid peroxidation. In some embodiments, the balloon 28 and/or the filament 30 may be pre-coated with antibiotics and/or anti-inflammatory agents, e.g., to act as a drug eluting balloon. For example, the balloon catheter may be utilized for other challenging body lumens, e.g., blood vessel, arteries, and other vasculature, ducts, tracts, body lumens, and the like.

FIGS. 5A-5E illustrate an exemplary embodiment of a method for capturing and delivering a substance into a Fallopian tube by the catheter 20 (e.g., see FIG. 4A). In the exemplary method, an inflation fluid may be introduced into the balloon 28, e.g., as shown in FIG. 5A and as indicated by arrow “A.” In some embodiments, the inflation fluid may be introduced via Y-connector 23 of the operating port 10 as a liquid, gas, or air, or combinations thereof, that inflates the balloon 28. The inflation fluid may be introduced in the balloon 28 substantially simultaneously with advancement of the sheath 32. The push wire 21 may be advanceable by a user in a direction as indicated by arrow “C” for everting the balloon 28. In some embodiments, the balloon 28 may be everted such that the balloon 28 extends distal of the distal end of the catheter. In some embodiments, the sheath may extend with the balloon 28 during eversion, to receive the substance “S”.

The distal end of the catheter 20 may be next aligned with a substance “S” to be delivered and may be captured by the balloon 28 as shown in FIG. 5B. In some embodiments, the substance may be delivered to the balloon and/or filament via injection, coating, and/or dipping. A blunt tip needle may be used for injecting a substance to the inner surface of the balloon. In some embodiments, the balloon may remain in an inverted position so that the substance is injected to the inner surface of the balloon. In some embodiments, the balloon may be at least partially everted before injecting the substance to the inner surface of the balloon. In other embodiments, the balloon may be at least partially everted and the substance may be delivered to the distal tip of the balloon. It is understood that when the balloon is re-inverted, the substance may at least be partially drawn in a proximal direction along the balloon surface.

In embodiments where the balloon and/or the filament is coated and/or dipped in a substance, the substance may be disposed in a container, e.g., a slide or a dish. The balloon may be at least partially everted, and the distal end may be positioned to contact the substance on the slide or dish. The balloon may then be re-inverted, such that the substance is at least partially drawn in a proximal direction on the inner surface of the balloon. It is understood that during re-inversion after capturing the substance, the substance may substantially coat the inner surface of the balloon and/or the filament. In some embodiments, the filament may be impregnated by a substance separately from the balloon by individually coating and/or dipping the filament on a slide or dish containing the substance.

As shown in FIG. 5C, the push wire 21 may be retracted in a direction as indicated by arrow C′ to pull the captured substance “S” held by the balloon 28 back into the protective sheath 32. The catheter 20 may be introduced into the uterus and at least partially to the Fallopian tube ostium “FT” (e.g., via hysteroscope) as shown in FIG. 5D.

When the catheter 20 is in a desired position relative to the Fallopian tube, as shown in FIG. 5E, the push wire 21 may be advanced in a direction as indicated by arrow “C”, thereby everting the balloon 28 such that the balloon 28 is extended into the Fallopian tube. As the balloon 28 everts, the substance “S” may be dispersed, ejected, or deposited in the Fallopian tube. In some embodiments, the substance S may be advanced out of the catheter 20 as a result of the balloon being everted, e.g., the inner surface of the balloon in an inverted position is advanced to become an outer surface of the balloon (see, e.g., FIG. 16).

The method of delivering a substance by the catheter 20 as well as exemplary embodiments including a lumen 35 as a hollow tube within the push tube 21′ may include introducing the catheter 20 into the uterus and to the Fallopian tube ostium “FT” (e.g., via a hysteroscope), pressurizing the balloon by a liquid, gas, or air, or combinations thereof, to inflate the balloon 28 either before or after the catheter 20 is introduced to the uterus. The push tube 21′ may be advanced in a distal direction to cause the balloon 28 to move between an inverted position and an everted position, for example, as an inner surface advances to a distal end and “unrolls” to an outer surface of the balloon, as shown in FIG. 16. A substance may be injected into the lumen 35 via the proximal opening 37 for dispersal out the distal end of the catheter, or via the distal end of the catheter (see e.g., FIGS. 5A-5E). The substance may be injected into the through lumen 35 when the balloon is in the inverted position, the everted position, during eversion, or combinations thereof. Substances to be introduced include, but are not limited to, radiopaque markers, radiopaque marking materials (e.g., including markers visible by way of CT scan, X-ray, or MRI scan), gels, chemotherapeutics, fertility therapeutics, antibiotics, protective substances, dissolvable objects, impermeable objects, radiation delivering objects or substances, etc.

When the balloon 28 is in an inverted position, for example as shown in FIG. 18A, a substance S may be held or stored in at least a portion of the lumen 35. In some embodiments, the substance S may be injected into the lumen 35 from a proximal end of the catheter, or a distal end of the catheter, or both. In some embodiments, a plurality of substances (e.g., “S1”, “S2”, . . . “SN”) may be injected into the lumen 35 for later dispersal in a Fallopian tube. It is understood that any number “N” of substances “S” may be included. In some embodiments, a first substance (e.g., “S1”) may be injected in the distal end of the catheter. If the first substance is tacky, or more viscous, a blockage may form in the catheter. A second substance (e.g., “S2”) may be injected into the lumen 35 from a proximal end of the catheter, which may act to lubricate, or to lessen the viscosity, of the first substance, so that both substances may be dispersed in the patient.

In embodiments, the substance S may be dispersed or ejected from the distal opening 36 of the lumen 35 into internal channel formed by the surface of the balloon 28 that is the inner surface when the balloon 28 is in an inverted position. The inner surface of the balloon may become coated by the substance S, which may contact the inner surface of Fallopian tube when the balloon in everted. For example, it may be desired to coat the Fallopian tube wall with a substance S as part of a treatment procedure or to protect a desired portion of the Fallopian tube wall from further treatment agents. In such a situation, the substance S may be injected into the lumen 35 when the balloon is in the inverted position. Accordingly, the inner channel of the balloon may be coated with the injected substance. Upon eversion of the balloon, the outer surface of the balloon, now coated in the substance S, may contact desired inner surfaces of the Fallopian tube, thereby transferring the substance S to the Fallopian tube walls for treatment, marking with a marking substance an area of interest, or protecting desired sections of the inner surface of the Fallopian tube, or combinations thereof.

When the balloon is in the everted position, for example as shown in FIG. 18B, a substance “S” may be dispersed or ejected from the distal opening 36 of the lumen 35 directly into the Fallopian tube. When the balloon 28 is in a fully everted position, the distal opening 36 of the through lumen 35 is generally the most distal portion of the catheter 20. Substances dispersed from the distal opening 36 of the lumen 35 of the push wire 21′ when the balloon 28 is in an everted position may be directly applied to the inner surface of the Fallopian tube. This may allow for precise delivery of substances to desired locations in the Fallopian tube.

In some embodiments, methods for delivery of substances to desired locations in a patient's Fallopian tube may be advantageous by minimizing, or limiting, a patient's exposure to a substance S to only desired areas, e.g., an area requiring treatment. For example, for a patient receiving cancer treatments, chemotherapeutic agents may be applied systemically, flushing the body with chemicals that may be harmful to healthy areas of the body. As described above, the catheter 20 may delivery a chemotherapeutic agent to desired locations in the Fallopian tube while avoiding other targeted areas, thereby potentially minimizing, or limiting a patient's exposure to the chemotherapeutic agent and reducing potentially harmful effects of the substance on otherwise healthy areas of the patient. In some embodiments, a marking substance may be delivered to a desired position in a Fallopian tube. The marking substance may be visible via CT-scan, X-ray, and/or MRI scan, to aid a medical professional in determining positioning in the Fallopian tube.

In some embodiments, the medical professional may desire to mark areas in the Fallopian tube where cell samples have been taken, or where cells are known to be abnormal, malignant, and/or cancerous, so that these desired areas may be monitored and/or treated. In some embodiments, an object may be delivered to a desired location within the Fallopian tube to block, minimize, or prevent spreading of injected/applied substances and/or cancerous cells. In some embodiments, the object may be impermeable. In some embodiments, the object may be bioabsorbable and/or biodegradable. In some embodiments, the object may be deliverable and/or retrievable by the catheter 20, e.g., such as an impregnated and/or coated suture or filament as described above. The object may be deliverable during balloon eversion, e.g., as the balloon unrolls. The object in contact with an inner surface of the balloon in the inverted position may be advanced out of the distal end of the catheter for placement in the Fallopian tube.

In some embodiments, a radiation delivering object and/or substances may be delivered to desired locations in the Fallopian tube for radiation treatment. In some embodiments, a dissolvable object such as polylactides may be delivered to a desired location in the Fallopian tube, such that the delivered substances may be released over an extended period of time as the dissolvable object dissolves, or medication is dispersed.

It is understood that exemplary embodiments of the delivery method as shown in FIGS. 5A-5E, as well as delivery via the lumen 35 in the push tube 21′ as shown in FIGS. 18A-18B, may be used independently or in connection with each other, or both, based on the substance or substances to be delivered and the desired location of delivery.

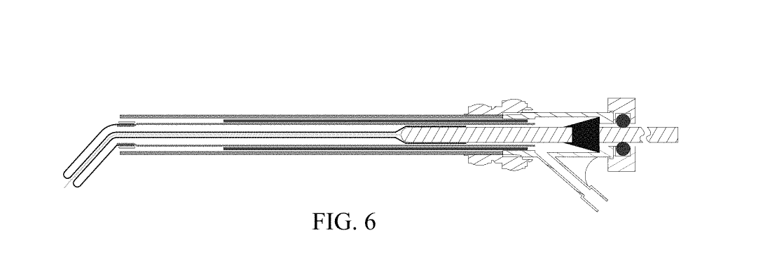

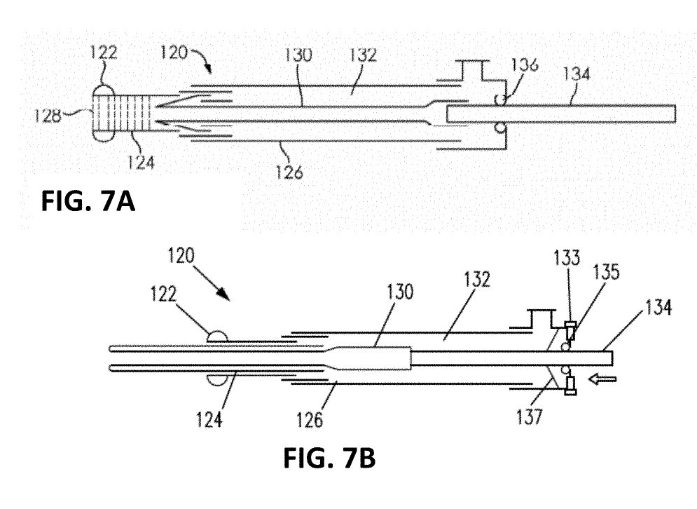

FIGS. 7A and 7B illustrate an exemplary embodiment of a ball tip catheter 120 in accordance with the present disclosure. A spherical ball 122 may be attached to the distal end of a spring tip 124 affixed to a tube, or catheter 126. It is understood that “tube” and “catheter” 126 may be used interchangeably. The spherical ball 122 may be provided to negotiate through a patient's UTJ to minimize and/or avoid inadvertent penetration through the UTJ sidewalls. The spring tip 124 may allow the distal end with the ball 122 to flex around corners and navigate through the UTJ. The spring tip 124 and spherical ball 122 may have an open lumen 128 extendable through the spring tip 124 and the spherical ball 122. The spherical ball 122 on the spring tip 124 may be approximately 0.8-1.0 mm in diameter, and the hollow spring tip 124 may have a length of approximately 1.5 cm and an outer diameter of approximately 0.6 mm. The hollow spring tip 124 may be formed of a metal (stainless steel or superelastic metal, e.g., Nitinol) coil spring sheathed on the outside with thin walled polymer heat shrink tubing, made of nylon, PET (polyethylene terephthalate), or similar material. In some embodiments, the spring tip 124 may be a metal coil spring co-extruded into a tubular polymer body. The hollow spring tip 124 may also be a flexible polymer tube, and in some embodiments may be made of nylon, Polyethylene terephthalate (PET), polyether block amide, or similar materials. An everting balloon 130 may lie inside the hollow spring tip 124. The everting balloon 130 may extend proximally inside the main lumen 132 of the introduction catheter 126 (e.g., a generally flexible tubular structure) or cannula (e.g., a generally rigid tubular structure).

The proximal end of the everting balloon 130 may be attached to a push rod, or push wire 134 passable through a seal 135 on the proximal end of the catheter 126 or cannula. In operational use on a patient, the flexible ball tip 122 may be manually advanced through the UTJ. Once passage of the flexible ball tip 122 and spring tip 124 through the UTJ occurs, the push wire 134 may be advanced through the seal 135 of the previously pressurized introduction catheter 126 or cannula. Advancement of the push wire 134 may cause a controlled eversion of the balloon 130 out of the hollow spring tip 124, through the length of the Fallopian tube.

The catheter 126 described above, and in greater detail below may be introduced into the uterus of a patient using an operating hysteroscope 200, an example of which is shown in FIG. 2. An operating hysteroscope 200 may include one or more working channels. One working channel may provide irrigation to distend the uterus and allow endoscopic visualization, and one or more additional working channels may allow instruments and/or catheters to be advanceable distally of the hysteroscope. The catheter 126 (e.g., FIGS. 7A and 7B) may be advanceable through the working channel of the operating hysteroscope, and may cannulate the proximal os of a Fallopian tube. The everting balloon 130 may be advanced through the proximal catheter 126 into the proximal portion of the Fallopian tube.

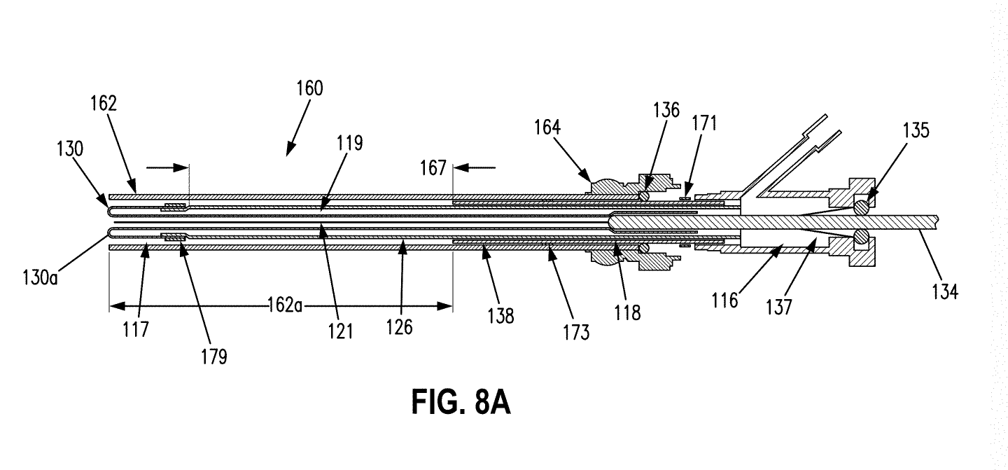

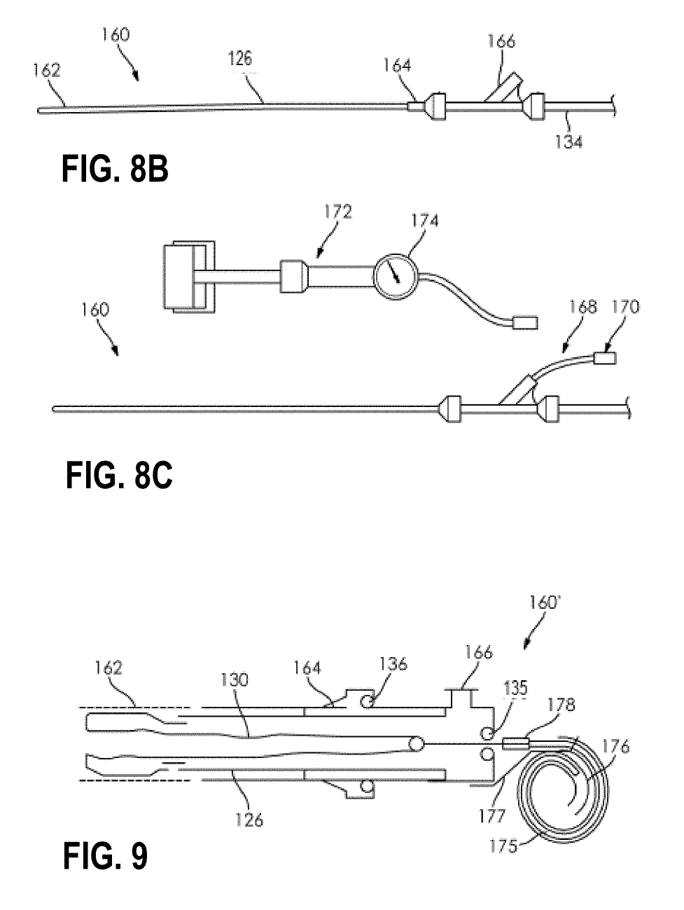

FIGS. 8A-8B illustrate a cross-sectional side view of a balloon catheter, or device, 160 in accordance with the present disclosure. In some embodiments, a balloon 130 may have an outer diameter of approximately 0.8-1.0 mm, and may have an initial everted length of approximately 1-3 cm, e.g., approximately 1.2-1.5 cm extending out of the distal end of the catheter 126 or cannula. The balloon 130 may be fully evertible into the Fallopian tube, e.g., extending approximately 7-12 cm. The balloon 130 may be securable to a distal end of the catheter shaft or tube 126, as indicated at reference numeral 117, and a push wire 134, as indicated at reference numeral 118. For example, the distal end 118 of the push wire 134 may form an end of the balloon 130. In embodiments, the balloon 130 may be bonded to the distal end 118 of the push wire 134. The push wire 134 may actuate the balloon 130 from an inverted position in the catheter 126 to an everted position when an interior of the balloon, between the catheter 126 and the balloon 130 and indicated by reference numeral 119, is pressurized. In embodiments, an everted position may include at least a portion of the balloon 130 extending beyond the distal end of the tube 126. In some embodiments, the balloon 130 may be initially partially everted and fixed to the catheter 126, forming a rounded end 130a. In some embodiments, the balloon 130 may be inflatable with fluid to a pressure of approximately 14-24 atm (206-353 psi).

The pressurized balloon 130 may have a rounded end 130a for atraumatic cannulation of the proximal os and advancement within the Fallopian tube and a degree of flexibility along the balloon 130 length. The balloon 130 may have sufficient column strength to allow the balloon 130 to be manually advanced through the UTJ, for example, with a push wire 134, under at least a partial pressure or no pressure. In some embodiments, the balloon 130 may be constructed of a thin-walled polymer material, such as polyethylene terephthalate (PET), polyethylene, Nylon, polymer, or a similar material. The balloon 130 may have a wall thickness from approximately 0.0001 to 0.001 inches and in some embodiments between approximately 0.00019 and 0.00031 inches.

In some embodiments, a first marker 171 may be disposed on at least a portion of catheter 126. The first marker 171 may be a preparation marker, indicating a desired position of the sheath knob 164. When the sheath knob is aligned with the first marker 171, the proximal end of the sheath 162 may be a reference point for the medical professional for balloon extension during preparation and initial cannulation of the balloon 130 into the Fallopian tube. In embodiments, at least a portion of the catheter 126, e.g., a proximal portion connected to the transparent portion 167, may be formed of a metal such as stainless steel, or other materials such as composites, or polymers, or combinations thereof. The first marker 171 may indicate to a user an appropriate location of male luer lock fitting, or sheath knob, 164 with respect to the balloon 130 within the sheath 162, so that the sheath 162 may be extended distally an initial length as a preparation step to cover, for example, approximately 10 to 20 mm length of everted balloon 130 that is used to access the proximal os before the balloon is completely everted.

In some embodiments markers may be incrementally spaced apart in known predetermined distances from each other such that a medical professional may use the markers as a visual counter or measuring device to verify an approximate length of balloon that has been everted. It is appreciated that any inner cannula or catheter described herein may include indicia as described for assistance in navigating patient anatomy.

In some embodiments, a second marker 173 may be disposed on the catheter 126, e.g., a metal portion 138, to indicate a desired location of sheath knob 164 to confirm that the sheath 162 covers the deployed everting portion (balloon, filament, etc.) during device removal into the hysteroscope 200. For example, the second marker 173 may be a retraction marker. This may allow the user to visualize and confirm that the balloon 130 is fully protected by the sheath 162 during the removal process to avoid loss of cells collected on the balloon and/or extended portion. When the hysteroscopic view is obscured, for example, by blood or tissue in the distension fluid, additional user visualization by the second marker 173 may be advantageous. The second marker 173 may be formed by the same techniques used to form the first marker 171. The second marker 173 may also be included on any inner cannula or catheter described herein.

In some embodiments, the balloon material may be treated to change the surface properties of an exterior surface of the balloon 130. Processes such as plasma or corona treatment may increase surface receptiveness to various substances that illustratively include subject cells, inks, coatings, adhesives, laminates, and paints, or combinations thereof. Surface treatment may enhance wettability creating a surface with hydrophilic properties, or discourage wetting creating a surface with hydrophobic properties. Surface treatment may be used to improve the adhesion properties of the balloon surface, to create a surface in which cells are more likely to adhere compared to an untreated surface.

Surface treatments may also be used to prepare the balloon surface for printing indicia on the surface, e.g., including PAD printing. PAD printing (also called tampography) is a printing process that may transfer a 2-D image onto a 3-D object. Indicia printed on the balloon surface may serve as preparation markers for the user. These preparation markers may allow the user to know the length of the balloon 130 prior to deployment of the balloon 130, thereby improving the ease of use of the device by eliminating the need for an outside measuring tool and improving the safety of the procedure by eliminating any guesswork or eyeballing on the part of the user.

In addition to marking for visualization purposes, the balloon 130 may also be treated with a process that increases surface area such as the application of a nanofiber or micropillar surface (e.g., including but not limited to ULTRA-WEB® from Corning), which may improve cell collection yield and/or retention compared to a balloon with little or no surface treatment. The filament, suture, or string 121 may include similar surface treatment features as a way to enhance cell collection and retention.

The balloon 130 may be translucent, optically transparent, or a combination thereof. In some embodiments, the balloon 130 may be at least partially opaque to enhance visibility during use. In some embodiments an opaque fluid may be mixed in the inflation fluid to control color of the balloon and to further enhance visibility of the balloon. The amount of the opaque fluid added to the inflation fluid may control the level of translucence or opacity of the balloon. In some embodiments, the fluid may be rendered opaque or otherwise detectable through the inclusion of colloidal or suspended particulate or microbubbles released within the fluid. Colloidal or suspended particulate operative herein include without limitation, polymethylmethacrylate, mica, barium sulfate, starch, and combinations thereof.

The length of the fully everted balloon 130 may extend to approximately 7-12 cm within the lumen (e.g., Fallopian tube), such that when fully everted, the balloon 130 may extend within the patient's Fallopian tube, following the successful advancement of at least a portion of length of everted balloon through the UTJ. Eversion of the balloon 130 may be performed in a controlled manner, e.g., by advancing a push wire 134 through a fluid tight seal 135, at the proximal end of the catheter 126. As described above, at least a portion 167 of the catheter 126 may be transparent or translucent, so that movement of the balloon 130 may be viewable through the hysteroscope through which the catheter 126 is inserted, thereby providing the user with a direct view of the insertion procedure. The catheter 126 may be constructed of polymers such as Nylon, polyether block amide, polyurethane, PET (polyethylene terephthalate), polyethylene, or polyvinyl chloride (PVC), with or without polymer or metal coil or braid reinforcement, or combinations thereof.

In some embodiments, a balloon 130, when everted at least partially out of the catheter 126 or cannula, may not remain straight. Rather, the balloon 130 may assume an undesired curved configuration, either a single “C” curve, or an “S” curve, that may be difficult to use to cannulate the proximal os of the Fallopian tube, and to advance the balloon through the UTJ. The extended length of everted balloon 130 may be straightened out or maintained straight by use of an outer sheath 162 that lies coaxial about the exterior of catheter 126 or cannula, and may assist in providing column strength and cover of the partially everted balloon tip. At least a portion of sheath 162 and/or catheter 126 may be transparent 167, e.g., 167 of FIG. 8A, so that movement of the balloon 130 may be viewable through the hysteroscope through which the catheter is inserted, thereby providing a user with a direct view of the insertion process. Similar to catheter 126, the sheath 162 may be constructed of polymers such as Nylon, polyether block amide, polyurethane, PET (polyethylene terephthalate), polyethylene, or polyvinyl chloride (PVC), with or without polymer or metal coil or braid reinforcement, or combinations thereof. The sheath may be alignable with respect to the catheter and/or the balloon, thereby providing column strength to the balloon. In response to cannulation of the balloon through the UTJ into the Fallopian tube, the sheath may support the balloon from outside of the proximal os of the Fallopian tube to minimize and/or prevent collapse as the balloon is further everted after navigating the UTJ. The sheath 162 may also protect the sample (e.g., cells) collected on the balloon and/or extended portion. For example, the sheath 162 may protect the balloon in an everted position after contacting an inner surface of the Fallopian tube. In some embodiments, the sheath 162 and balloon with or without extended portion may be retracted coaxially with the inner lumen of the sheath to extend the sheath over the everted balloon, and extended portion, if included, subsequent to cell collection. In some embodiments, the sheath 162 may remain stationary relative to the balloon 130 and/or the catheter 126, so that the balloon 130 is received in the sheath 162 subsequent to cell collection. As the balloon 130 is withdrawn into the sheath and removed from the patient, the sheath may protect the cells to minimize and/or prevent loss of the sample collection by providing a barrier from distention fluid in the uterus or irrigation fluid in the Fallopian tube or uterus. For example, the balloon subsequent to cell collection may otherwise be exposed to environmental conditions that may render the sample collection unusable, and/or otherwise wash the cells from the balloon and extended portion.

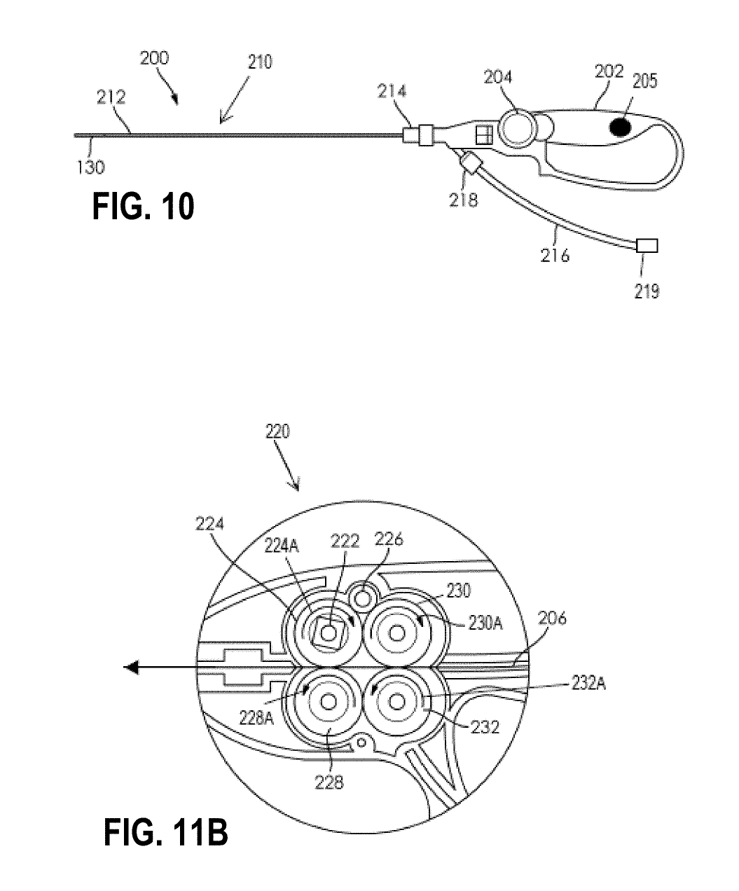

FIG. 16 illustrates an exemplary embodiment of a linear eversion of a balloon 130 in accordance with the present disclosure. In embodiments, one end of the balloon may be fixed to inner cannula/tube at point X (e.g., reference numeral 117 as illustrated in FIG. 8A) and the other end of the balloon may be movable at point Y (e.g., reference numeral 118 as illustrated in FIG. 8A). The balloon 130 may evert from the position shown in Step 1 to the position shown in Step 2 to the position shown in Step 3. In the eversion process, points A, B, and C move towards the left side of the diagram, e.g., extend distal of the distal end of the device 160. As the balloon 130 unrolls/everts at/toward the left side of the diagram, point A may move from the inside surface of the balloon to the outside surface. In practice, the balloon 130 that has been partially, or initially, everted during the preparation step may be advanced further into the proximal end of the Fallopian tube. Further eversion (extension) of the balloon (in total up to the full length of the Fallopian tube, approximately 7-12 cm) may be accomplished by further rotation of a drive wheel 204 (see FIG. 10). The balloon 130 may then be deflated by relieving pressure in the inflation device. The balloon 130 may then be retracted from the Fallopian tube. Because the Fallopian tube is a potential space, the Fallopian tube tissue may tend to collapse around the balloon. Because the balloon fills the Fallopian tube, the balloon surface area may be substantially equivalent to the surface area inside the Fallopian tube. This surface area may optimize tissue collection from the inside of the Fallopian tube. While deflation of the balloon may be desirable prior to retraction, it may be possible in some embodiments to retract a balloon/extended portion from a Fallopian tube without first deflating the balloon and still retaining cells collected thereon. For example, the balloon 130 in an inflated and/or a deflated state may be retracted within the sheath 162 while retaining a sufficient amount of cells on the surface of the balloon 130 for testing. Alternatively, the balloon may be repeatedly inflated and deflated while extended in the Fallopian tube, so that each time the balloon contacts Fallopian tube walls, more cells may be collected and/or retained by the balloon.

In some embodiments, to further aid tissue collection, wrinkles or other surface features may be added to the surface of the balloon. Wrinkles may form as the balloon deflates to create multiple edges and/or overlapping material, to aid in cell collection. Edges may work in a manner similar to the edges of a curette or edges of jaws in a biopsy forceps. Similar to these features on other collection devices, edges formed by the wrinkled balloon may focus a contact force on the anatomical wall in order to collect cells.

The balloon deployment device in accordance with the present disclosure may then be removed from the working channel of the hysteroscope and from the patient. Once the device is removed from the patient, cells may be removed from the balloon by dipping the balloon and/or the extending portion (if used) into a cytopreservative and stirring in order to agitate the cells. Alternatively, balloon, extending portion, and/or sheath may be cut off and placed into a cytological preservative. In some embodiments a sheath may be extendable and deployable over the balloon as the balloon is deflated and removed to protect tissue samples collected on the balloon surface.

FIG. 9 illustrates a cross-sectional side view of a balloon catheter 160′ including a superelastic push rod, or a push wire 175 and spiral carrier 176. The spiral carrier may minimize and/or eliminate the need to extend the push wire backwards, e.g., outside of a handle, for the full length of the push wire in accordance with embodiments of the disclosure. The push wire 175 may be constructed of a superelastic material such as Nitinol (nickel-titanium compound) wire. At least a portion of a length of push wire 175 may be coiled into a spiraling tubular carrier 176, which may be made of polyethylene or polytetrafluoroethylene (Teflon). The outer spiral diameter of the carrier may be approximately 8 cm, rendering the proximal operating length of the catheter handle much more compact. The spiral carrier 176 may be attached to a proximal seal 135 on the catheter by a flexible strap 177. In some embodiments, the flexible strap 177 may be constructed of polymer or silicone rubber material. In some embodiments the push wire 175 may have a diameter of approximately 0.025″, or some other thin diameter, which may be disadvantageous for purposes of gripping the wire and push it forward through the seal 135. A flexible grip 178 may be included that slides freely on the push wire 175, but upon compression between the thumb and forefinger, may provide a grip for push wire 175 advancement. The flexible grip 178 may be an elliptical cross-section frame that may be made of polyvinyl chloride, silicone rubber, or combinations thereof, or similar flexible compound. In some embodiments, the flexible grip may have inner dimensions of approximately 2 cm in length, 1 cm in width, and 3 mm in height, and may have a wall thickness of approximately 2 mm. Holes in the proximal and distal faces of the grip may be a slip fit with the push wire 175.

FIG. 10 illustrates an exemplary embodiment of a balloon catheter 200 configured with handle 202. In embodiments, the handle 202 may be included in the device 160 as illustrated in FIG. 8A. The handle 202 may house a gear mechanism 220 (see FIGS. 11A-11B), also referred to herein as an actuator. The handle 202 may be in mechanical communication with a push wire 134, 206 and may control actuation of the push wire 134, 206, which in turn may control actuation of the balloon 130 between an inverted position and an everted position. Handle 202 may include a drive wheel 204 for advancement and retraction of the push wire 134, 206, in which the balloon 130 may evert linearly (e.g., gradually unfold or unroll from the inside out.). The drive wheel 204 may be made of polymer material including but not limited to ABS. The outer edge of the drive wheel 204 may include notches, or a knurl pattern, to facilitate gripping the wheel during operation of the catheter 200. The outer edge of the drive wheel 204 may include multiple features shaped like arrow heads that facilitate gripping and/or may indicate correct direction of travel of the drive wheel. A top surface of the drive wheel 204 may have an arrow molded into it for indication of a correct direction in which to turn in order to evert the balloon. The opposite side of the drive wheel 204 may include a square boss 222 insertable into a drive gear 224. In some embodiments, the gear mechanism 220 may include a step-down gearing that provides a reduced amount of extension of the push wire 134, 206 relative to a given rotational distance travelled by the drive wheel 204 (i.e., the drive wheel 204 must be turned more of a distance to accomplish the same extended length of balloon everted, than if step-down gears were not included or a different ratio of step-down was included). The resultant effect may be to have a finer control over the eversion of the balloon 130 as the drive wheel 204 is turned.

The catheter 200 may retain the balloon 130 in a shaft 210 (which may at least partially be formed of a stainless steel tube and/or a Nylon tube), a sheath 212, and/or a sheath knob 214. For balloon advancement, the balloon 130 and shaft 210 may be pressurized with an inflation device (such as inflation device 172 of FIG. 8C) that is attachable to an extension tube 168, 216, or to luer 218, of the handle 202 (see FIGS. 11A-11B). In some embodiments, luer 219 may be attachable to the extension tube 168, 216. In some embodiments, the push wire 206 may extend proximally from the handle 202 and may include a luer 219′. The push wire 206 may be hollow, thereby allowing for delivery of one or more substances through the device to a desired tissue location. In some embodiments, the push wire may include a single tube, connecting a balloon at a distal end and a luer at a proximal end, such that one or more substances may be easily and efficiently delivered to a desired tissue location.

Once the catheter device 200 is pressurized, a user may rotate the drive wheel 204 causing a push wire 134, 206 to advance. Although in some embodiments, the balloon 130 may evert under pressurization without a drive wheel advancement of the push wire 134, 206, it is understood that the drive wheel may allow for smooth, slow, controlled advancement of the balloon, thereby minimizing or avoiding potential perforation of the Fallopian tube. The sheath knob 214 may allow the sheath 162, 212 to be used as an introducer as the sheath 162, 212 locks onto the body of the catheter 126, 210. The sheath knob 214 may be compliant enough to allow the user to move the sheath 162, 212 when needed, for example to the pre-extended portion of the balloon and to move the pre-extended portion of the balloon into the Fallopian tube. In embodiments, the sheath knob 214 may be tight enough such that unintended balloon or catheter movement may be minimized and/or prevented.

FIG. 11A is a cross-sectional view of the handle portion of FIG. 10, and FIG. 11B is a detail view of an exemplary embodiment of an internal handle gear mechanism 220 in accordance with the present disclosure. The handle 202 may also have an extension tube 168, 216 that is attached to a luer 218 in the handle body, e.g., for attaching one or more additional tools or devices such as inflation device 172 (see also FIG. 8C). The gear mechanism or actuator 220 may be in mechanical communication with the push wire 134, 206, and may control actuation of the push wire 134, 206, which in turn may control actuation of the balloon 130 between the inverted position and the everted position. In some embodiments, the gear mechanism or actuator 220 may include a plurality of gears operating enmeshed to have a step-down ratio. According to various embodiments, the handle gear mechanism 220 may include a drive wheel 204, which allows controlled actuation of the gear mechanism 220 and single user operation.

In some embodiments, to provide feedback to the physician regarding the end of balloon deployment, the internal handle gear mechanism 220 or actuator may include a limit mechanism on the gears for limiting the advancement of the push wire and/or a unidirectional balloon movement. In some embodiments, the limit mechanism may include at least one of a hard stop, a gear jam, a rack and pawl gear, a linear gear, or a drop key-click in mechanism. At a predefined maximum extension, a pawl 242 may engage with one or more gears (e.g., gears 224, 228, 230, 232) as shown in FIG. 11E, to form a gear jam. The pawl 242 may be activated to stop further advancement of the balloon 130. In embodiments, the pawl 242 may be any mechanism configured to engage with one or more gears. For example, at a predefined push wire extension, the pawl 242 may rotate around a pivot point to engage with one or more gears, causing a jam and preventing further rotation. Alternatively, a rack and pawl gear, a linear gear, or a drop key-click-in mechanism (FIG. 11D) may be employed to stop advancement of the balloon and in some embodiments may be disposed in the handle (see FIG. 11A, detail “F”). Referring to FIG. 11C, an exemplary ratchet mechanism for linear motion is shown. Ratchets are mechanisms that serve to limit motion to only one direction. A ratchet may have three main parts: a linear gear rack 233, a pawl 235 (e.g., a “click”), and a base or mount 237. The edges on one side of the teeth 239, 239′ on the linear rack may have a steep slope while the other edges of the rack's teeth may have a moderate or gradual slope. For example, edges on one side of the teeth 239, 239′ may be steeper than edges on another side of the teeth 239, 239′. In some embodiments, a steeper slope may have an angle of approximately 60°-90°, e.g., as indicated at 239b, 239b′, and a more moderate slope may have an angle of approximately 10°-50°, e.g., as indicated at 239a, 239a′. The pawl 235 may contact the linear gear rack 233. When the linear rack is linearly moved in a first direction, the pawl 235 may slide over the teeth 239 without restricting the natural motion of the device. When the direction of motion is reversed to a second direction, the pawl 235 may contact the steep slope on the gear tooth 239 to impede motion. The pawl 235 may be biased downward by a spring into the linear gear rack 233. In some embodiments, a spring, e.g., a torsional spring, may be disposed at a pivot point 236, e.g., at a first end of pawl 235, for pivotable rotation of the second end of the pawl 235. In some embodiments, a spring, e.g., a linear spring, may be disposed at a second end of the pawl 235, as indicated by reference numeral 223, to bias the pawl 235 towards the gear teeth 239. The linear gear rack 233 and pawl 235 may be typically mounted in a fixed relationship to one another on a mount 237, with the rack sliding in relation to the mount and the pawl 235 having a pivot connection to the mount. In some embodiments, the device may include a manual knob or push button switch to overcome the spring bias on the pawl 235 to allow for the lifting of the pawl 235 from the set of teeth on the linear gear.

A limit may be set on the ratcheting action of the linear gear rack 233 in the gear mechanism 220 of FIGS. 11A-11B to set a limit on the advancement of the push wire 206, e.g., as shown at “F”. During advancement of the push wire 206, a pawl 238 may be biased toward from linear gear rack 233 as shown in detail “F” of FIG. 11A. In some embodiments, the pawl 238 may be pivotal about a point, as indicated by reference numeral 221. Linear gear rack 233 may be directly attached to the end of the push wire 206 away from the balloon 130 in the handle 202. Advancement of the push wire 206 may be automatically stopped when pawl 238 meets stop 243, which may be greater in height than teeth 239′. A manual knob or push button switch 205 as illustrated in FIG. 10 may be actuatable by a user to overcome a spring bias on the pawl 238 to allow for the lifting of the pawl 238 from the linear gear rack 233 and for retraction of the push wire 206 and the attached balloon 130. In FIG. 11D, in another embodiment different from the ratcheting action of the linear gear rack 233, the push wire 206 may be continuously and smoothly advanced and coiled around deployment wheel 245 until pawl 235 reaches and engages with detent 247 to stop further advancement of the balloon 130. In FIG. 11E, the pawl 235 may act as a gear jam when an extension limit of the balloon 130 is reached.

The sequence of steps used to enter and track through the Fallopian tube may be described with the embodiment of FIG. 8A. When it is desired to cross the UTJ with a length (e.g., approximately 15 mm) of an everted balloon 130, the outer sheath 162 may be placed in apposition with the proximal os of the Fallopian tube, without entering the proximal os. The outer sheath 162 may support the initial length of everted balloon 130 until it enters the proximal os. A portion of the balloon, e.g., a short length, of pressurized everted balloon 130 exiting the supportive outer sheath 162 may have sufficient column strength to be manually advanceable through the UTJ, whereas an unsupported length (e.g., without the sheath) of the everted balloon 130 may not contain sufficient rigidity by itself. As such, an everted/everting balloon 130 may buckle upon attempted advancement through the proximal os and UTJ without a sheath. In some embodiments, crossing the UTJ with a length of the everted balloon (e.g., 15 mm), may occur. This initial cannulation length may support keeping the Fallopian tube open even if a spasm occurs, which may occur in this area of the Fallopian tube. It is also understood that other cannulation lengths may be utilized to maintain an open Fallopian tube.

In some embodiments, the sheath 162 may be compatible with standard hysteroscopes having a working channel, e.g., 5F. A sheath 162 may be used in an exemplary system as a balance to provide a wall thickness great enough to impart sufficient column strength to the sheath and thin enough to maintain a sheath inner diameter large enough to accommodate the balloon 130. This balance may improve cell collection efficiency, e.g., by having an inner diameter sufficient to retain the balloon 130 without inadvertently removing (scraping) cells from the balloon surface. It is understood that the balloon 130 may be retained within the sheath 162 in an inflated state and/or a deflated state.

As mentioned, a male luer lock fitting, or sheath knob, 164 including a Tuohy-Borst seal 136 connector may be included at the proximal end of the sheath 162. A Tuohy-Borst adapter that includes seal 136 is a medical device used for creating seals between devices and attaching catheters to other devices. The Touhy-Borst seal 136 may be tightened to have a slip fit with the catheter or cannula holding the sheath 162 in place. The sheath knob 164 may mate with a female luer lock fitting, if present, at an instrumentation port, on the working channel of the hysteroscope 200. Referring back to FIG. 2, the male luer lock or sheath knob 164 may be connectable to the instrumentation port 230 so that the catheter 126 and/or sheath 162 may move with the hysteroscope 200. In some embodiments, the instrumentation port 230 may further include a seal for the catheter 126 to extend through. When these respective luer fittings are connected, the tip of the sheath 162 may protrude out of the distal end of the hysteroscope, e.g., approximately 2-3 cm. The sheath 162 may also protect a portion of a balloon 130 everted during device preparation (e.g., a length approximately 1.5 cm) from injury as the catheter 126 is advanced through the working channel of the hysteroscope. A stainless steel tube, e.g., hypotube 138, may be at least a portion of the inner cannula 126 to provide sufficient rigidity and/or column strength to minimize or prevent kinking or collapse of a portion 605 protruding from the proximal end of the hysteroscope working channel, as shown in FIG. 6. In some embodiments, the hypotube 138 may be sized having approximately 0.050″ OD×0.004″ wall thickness for sufficient rigidity.

FIG. 8C illustrates a balloon catheter 160 of FIG. 8A with a tubing reservoir, or extension tube 168, and inflation device 172 in accordance with an exemplary embodiment of the disclosure. It is understood that in some embodiments the extension tube 168 may be similar to extension tube 216 as shown in FIG. 11A. The extension tube 168, 216 may be configured to withstand pressurization. Pressurization of the balloon 130 by fluid injection may be performed using a syringe device, such as the exemplary inflation device 172. Rotation of a threaded plunger shaft through a releasable lock may increase and maintain pressure in the inflation device 172, while a pressure gauge 174 provided with the inflation device 172 may allow for control of input pressure. In some embodiments, the balloon catheter 160 may provide for a one-person operation of the device. A length of pressure tubing, or extension tube 168, 216, may be added between the inflation device 172 and the inflation port 166 on the device. The extension tube 168, 216 may be constructed of polymers such as polyurethane or polyvinyl chloride (PVC), with or without polymer or metal coil or braid reinforcement. The extension tube 168, 216 may contain an amount of intrinsic elasticity, while the everting balloon may be generally inelastic. At full pressurization of the balloon 130, the extension tube 168, 216 may impart a fluid capacitance to the system. A small volume of fluid may be containable in the everted balloon, and this volume may be further subtracted by the volume occupied by the push wire 134 (e.g., which moves into the balloon 130 as it is being everted). The resultant everted balloon volume may be small compared with the larger volume in the pressure tubing 168, which may allow the balloon 130 to evert to its full length without significant decrease in pressure, once the balloon catheter 160 has been pressurized.

As described above with respect to FIGS. 8A-8C, the everting balloon 130 may be extendable a total distance of approximately 7 cm distal to the tip of the catheter, in order to pass through the entire length of the Fallopian tube. The everting balloon 130 may form a toroidal shape at the end 130a as it exits the catheter tip, and the everted portion may include a double walled configuration. A toroidal shape may be an atraumatic shape for minimizing or avoiding damage during extension into the Fallopian tube. Thus, for example, the push wire 134 advances forward a distance of approximately 14 cm in order to yield an everted balloon length of 7 cm. This length of push wire may initially extend backwards from the proximal end of the catheter 126, directly into the face of the operator, making its use cumbersome. The push wire may also be susceptible to contamination of the sterile device due to its length as it may extend into a physician's working space during a procedure. For example, the proximal end of the long push wire 134 may contact the physician's face or surgical mask during use. Therefore, it may be desirable to provide a push wire system that does not have to extend backwards for the full length of the push wire 134. The superelastic push wire 175 and carrier design of FIG. 9 and the balloon catheter 200 configured with handle 202 of FIG. 10 may contain the push wire and minimize and/or avoid the need to extend the push wire back towards the user.

FIG. 12 illustrates a cross-sectional side view of an exemplary everted balloon catheter 180 including a tube 182 having diameter smaller than the inflated diameter of the everting balloon 130 for insertion into the patient's UTJ in accordance with the present disclosure. The tube 182 may straighten a portion of the balloon tip 163. In some embodiments, the tube 182 may extend distally to the tip of the cannula. In embodiments, the tube 182 may have an approximately 0.0005″-0.001″ wall thickness (e.g., being a “thin-walled” tube), and may extend approximately 1.5 cm distal to the tip of the cannula. The tube 182 may have a thickness and resiliency sufficient to support the balloon 130, to maintain a position of the balloon tip 163 (e.g., maintain a straight position). In some embodiments, the tube 182 diameter may be smaller than the balloon 130 diameter, so that the balloon 130 may retain flexibility and compressibility. This flexibility may be beneficial to allow the balloon 130 to be advanced through the UTJ. In some embodiments, the balloon 130 may include the tube 182 to support and/or straighten the balloon. In embodiments, the tube 182 may have a 0.033″ OD×0.001″ wall×1.5 cm long.

FIG. 13 illustrates a cross-sectional side view of an everted balloon catheter 190 including one or more flexible polymer monofilament string and/or filament 192 as an extending portion attached to the distal end of the cannula or catheter 126. The strands 192 may extend into everting balloon 163, thereby supporting and keeping the tip straight for insertion into the patient's UTJ in accordance with an embodiment of the present disclosure. In some embodiments, the one or more flexible polymer monofilament string and/or filament 192 may extend into the balloon 163 (e.g., approximately 1.5 cm). The monofilament 192 may be formed of nylon, polypropylene, or other flexible polymer material, or combinations thereof. The monofilament strands may have a diameter of approximately 0.006″-0.012″. In some embodiments, the balloon 130 may have approximately a 0.033″ (0.8 mm) OD with a 0.008″ diameter monofilament 192 inside an approximately 1.5 cm long everted balloon tip.

In embodiments, a portion of the everted balloon may be treated with fluoropolymer, silicone, and like material coatings, or combinations thereof, lubricating the surface at the lead portion of the balloon catheter, which may enter the constricted portions of the Fallopian tube (e.g., the UTJ).

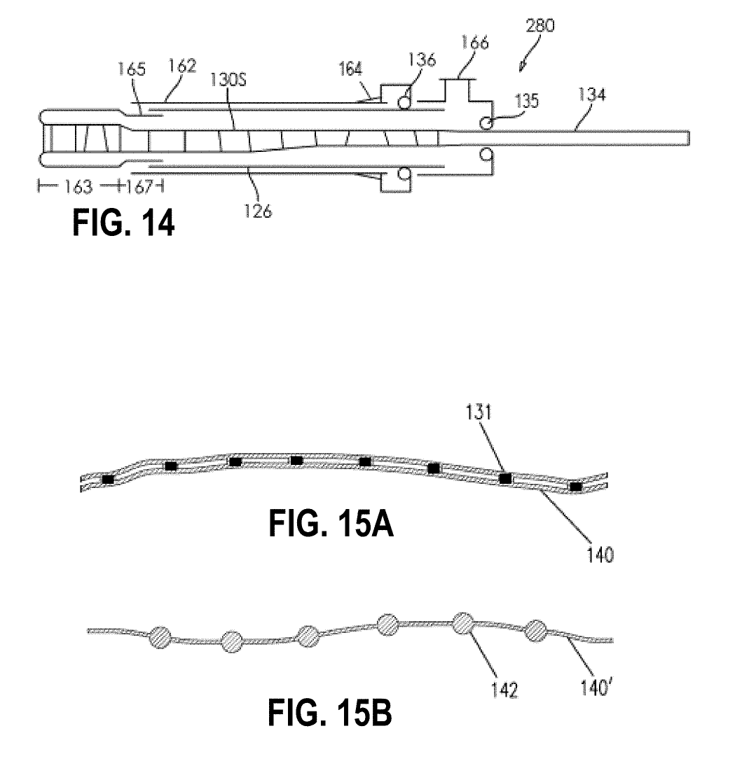

FIG. 14 illustrates a cross-sectional side view of a balloon catheter 280 configured with striped balloon 130S in accordance with an exemplary embodiment of the present disclosure. As shown in FIG. 14, the indicia 131 of the striped everting balloon 130S may be coupled with a transparent distal section 167 of the cannula or catheter 126 to provide visual feedback of balloon eversion. In some embodiments, the indicia may be pad printed or scribed with an indelible marker in a highly visible color. In some embodiments, the indicia 131 may be approximately 1 mm wide, spaced approximately in 0.5 cm increments along the entire length of the balloon. Pad printing (also called tampography) is a printing process for transferring a 2-D image onto a 3-D object. Other patterns may be used instead of, or in addition to, indicia 131 on the surface of the balloon 130S. For example, indicium 131 on the balloon 130S may be spaced apart (e.g., approximately 0.5 cm), and dots may also be added in the remaining intervals between the indicia 131. Each indicium 131 that comes into view in the transparent distal section 167 may indicate a successful eversion of a length of a balloon 130S (e.g., 0.25 cm, as the push wire is advanced approximately twice the length for a corresponding approximate length of balloon eversion (e.g., 0.5 cm). Indicia 131 of different thicknesses may be used, as well as different colored indicia, or a different number of indicia, or combinations thereof, in the same fashion described for the stripe and dot combination. In some embodiments, color coded sections may be added to the balloon 130S to indicate the extent of the balloon eversion.

Additional embodiments of feedback markers, which may be externally visible to the physician on the outside of the patient's body, for the extent of positive balloon eversion. In some embodiments, a knotted string or suture as an extending portion may be adhered to the distal end of the push wire or tip of the balloon, and may be spaced in known increments to provide tactile feedback as to balloon eversion progress. The knotted string or sutures may allow for visualization of the forward movement of the balloon as it is everted. The knotted string or sutures may be radio opaque. In some embodiments shown in FIG. 15A, a string 140 may be pad printed with indicia 131 in a similar manner to the balloon as noted above in FIG. 14, FIG. 15B illustrates a string 140′ with a series of knots or sutures 142. The balloon 130 may be at least partially transparent to enhance visibility of string, indicia, knots, or sutures.

Additional feedback mechanisms may include filling the balloon 130 with agitated saline and visualizing air bubbles with ultrasound, and a sinusoidal pattern for the balloon, where the distances between maximums of a sinusoidal wave define an incremental distance of balloon eversion.

Navigation within the Fallopian tube and the indication of a clear path or obstructions may be provided by release of microbubbles from the tip of the balloon or from the distal end of the tube that the balloon everts from. Travel of the microbubbles may be trackable using imaging, such as ultrasound, to ascertain where a clear path exists. In instances of an obstruction 251, e.g., an occlusion or a constriction, the microbubbles may bunch up, or congregate, when the microbubbles are impeded. In response to detecting a grouping of microbubbles, a medical professional may be able to ascertain an obstruction. FIG. 17A illustrates a release of a stream of microbubbles 249 from a tip of the balloon 130 in the Fallopian tube 1 where no constrictions or obstructions are present, as evident by the steady continuous line of microbubbles 249. In some embodiments, microbubbles may be delivered through a lumen 54 of a balloon, as shown in FIGS. 18A-18B. The frequency or spacing of the microbubbles 249 may be controllable for finer measurements than with an air source that is modulated on or off, where the air is introduced to the fluid injected into the balloon 130. FIG. 17B illustrates a Fallopian tube 1 with a tubular constriction or obstruction 251, where the tubular constriction or obstruction 251 may impede a flow of microbubbles 249 and the microbubbles 249 begin to congregate, or bunch up, at the point of the constriction or obstruction 251. The bunching of the microbubbles 249 may provide a visual indication to the user where the constriction or obstruction 251 is in the Fallopian tube 1. In response to a detected obstruction 251, a medical professional may perform additional imaging, such as ultrasound, to determine where the balloon stopped.

Any patents or publications mentioned in this specification are herein incorporated by reference to the same extent as if each individual publication was specifically and individually indicated to be incorporated by reference. The foregoing description is illustrative of particular embodiments of the disclosure, but is not meant to be a limitation upon the practice thereof.

Numerous specific details have been set forth herein to provide a thorough understanding of the embodiments. It will be understood by those skilled in the art, however, that the embodiments may be practiced without these specific details. In other instances, well-known operations, components, and circuits have not been described in detail so as not to obscure the embodiments. It can be appreciated that the specific structural and functional details disclosed herein may be representative and do not necessarily limit the scope of the embodiments.

Some embodiments may be described using the expression “coupled” and “connected” along with their derivatives. These terms are not intended as synonyms for each other. For example, some embodiments may be described using the terms “connected” and/or “coupled” to indicate that two or more elements are in direct physical or electrical contact with each other. The term “coupled,” however, may also mean that two or more elements are not in direct contact with each other, but yet still co-operate or interact with each other.

It should be noted that the methods described herein do not have to be executed in the order described, or in any particular order. Moreover, various activities described with respect to the methods identified herein can be executed in serial or parallel fashion.

What is claimed is:

1. A system for delivering one or more substances into a Fallopian tube of a patient, comprising:
a balloon catheter including a tube having a distal end, a balloon having a first end coupled to the distal end of the tube, and a push wire having a distal end coupled to a second end of the balloon, the balloon being movable between an inverted position and an everted position by actuation of the push wire, the balloon catheter being configured to:
receive the one or more substances such that the one or more substances are retained by the balloon, the push wire, or both; and
advance the push wire to evert the balloon such that the balloon extends distally of the distal end of the tube;
whereby in the everted position the one or more substances are delivered into the Fallopian tube.

2. The system according to claim 1, wherein the balloon catheter receives the one or more substances at the distal end of the tube and the first end of the balloon when the balloon is in the inverted position.

3. The system according to claim 1, wherein the push wire is hollow, the one or more substances being received into the proximal end of the push wire.

4. The system according to claim 1, wherein the balloon catheter receives a first substance at the distal end of the tube and the first end of the balloon, and wherein the push wire being hollow such that a second substance is received into the proximal end of the push wire.

5. The system according to claim 1, wherein the one or more substances at least partially coat an inner surface of the balloon when the balloon is in the inverted position.

6. The system according to claim 1, wherein the balloon catheter includes a filament attached to the distal end of the push wire, or the second end of the balloon, or both, the filament being configured to absorb at least a portion of the one or more substances.

7. The system according to claim 6, wherein the filament is configured to receive a first substance, and the balloon is configured to receive a second substance different from the first substance.

8. The system according to claim 1, wherein the one or more substances are at a temperature different than a temperature of the patient.

9. The system according to claim 1, wherein the one or more substances is any of a radiopaque marker, a radiopaque marking material, a gel, a chemotherapeutic, a fertility therapeutic, an antibiotic, an anti-inflammatory agent, a tissue protecting substance, a dissolvable object, an impermeable object, or a radiation delivering object, or combinations thereof.

10. A method for depositing one or more substances in a Fallopian tube of a patient, comprising:
receiving the one or more substances into a balloon catheter, the balloon catheter including a tube having a distal end, a balloon having a first end coupled to the distal end of the tube, and a push wire having a distal end coupled to a second end of the balloon, the balloon being movable between an inverted position and an everted position by actuation of the push wire, wherein the one or more substances are retained by the balloon, the push wire, or both;
advancing the push wire to evert the balloon to the everted position such that the balloon extends distally of the distal end of the tube;
depositing the one or more substances into the Fallopian tube in the everted position of the balloon.

11. The method according to claim 10, further comprising:
prior to receiving the one or more substances into the balloon catheter:
pressurizing the balloon in the inverted position by an inflation fluid to inflate the balloon; and
everting the balloon such that at least a portion of the balloon is extended distally of the distal end of the tube; and
after receiving the one or more substances into the balloon catheter, retracting the push wire such that the balloon is re-inverted and positioned proximal of the distal end of the tube such that the one or more substances are retained by the balloon.

12. The method according to claim 10, wherein the balloon is positionable within a sheath such that the balloon is extendable from a distal end of the sheath during eversion, and supports the balloon during re-inversion.

13. The method according to claim 10, wherein the balloon catheter includes a filament attached to the distal end of the push wire, or the second end of the balloon, or both, the filament being configured to absorb at least a portion of the one or more substances.

14. The method according to claim 13, wherein the filament is configured to receive a first of the one or more substances, and the balloon is configured to receive a second of the one or more substances different from the first substance.

15. The method according to claim 10, wherein the push wire is hollow, the one or more substances being received into the proximal end of the push wire.

16. The method according to claim 10, wherein the one of more substances is any of a radiopaque marker, a radiopaque marking material, a gel, a chemotherapeutic, a fertility therapeutic, an antibiotic, an anti-inflammatory agent, a tissue protecting substance, a dissolvable object, an impermeable object, a radiation delivering object, or a combination thereof.

17. The method according to claim 10, wherein the balloon catheter receives the one or more substances at the distal end of the tube and the second end of the balloon when the balloon is in the inverted position.

18. The method according to claim 10, wherein the balloon catheter receives a first of the one or more substances at the distal end of the tube and the second end of the balloon, and wherein the push wire being hollow such that a second of the one or more substances is received into the proximal end of the push wire.

19. The method according to claim 10, wherein the one or more substances at least partially coat an inner surface of the balloon when the balloon is in the inverted position.

20. The system according to claim 10, wherein the push wire is advanced to position the balloon in the everted position prior to receiving the one or more substances in the balloon catheter such that a surface of the balloon contacting an inner surface of the Fallopian tube in the everted position is free of a coating of the one or more substances.
